(19) United States
(12) Patent Application Publication
(10) Pub. No.: US 2018/0325891 A1
(43) Pub. Date: Nov. 15, 2018

(54) OCTAHYDRO AZADECALIN FORMULATIONS

(71) Applicant: Corcept Therapeutics, Inc., Menlo Park, CA (US)

(72) Inventors: Ian Scott, Dublin, CA (US); Wei Tian, Livingston (GB); Jenifer Mains, Livingston (GB); Graeme Johnston, Livingston (GB)

(21) Appl. No.: 15/976,665

(22) Filed: May 10, 2018

Related U.S. Application Data
(60) Provisional application No. 62/504,461, filed on May 10, 2017.

(51) Int. Cl. A61K 31/4745; A61K 9/48

(52) U.S. Cl. CPC A61K 31/4745; A61K 9/4866; A61K 9/4858

(57) ABSTRACT

Applicant provides formulations and pharmaceutical compositions containing glucocorticoid receptor modulators (GRMs) and suitable for treating diseases including Cushing's syndrome, prostate cancer, breast cancer, and other cancers, liver diseases, depression, dementia, stress disorders, and substance abuse disorders. The GRM may be a non-steroidal GRM, and may be an octahydro azadecalin GRM. In particular, the GRM may be CORT125281, which is: ((4aR,8aS)-1-(4-fluorophenyl)-6-((2-methyl-2H-1,2,3-triazol-4-yl)sulfonyl)-4,4a,5,6,7,8,8a,9-octahydro-1H-pyrazolo[3,4-g]isoquinolin-4a-yl)(4-(trifluoromethyl)pyridin-2-yl)methanone. Methods of treating diseases including Cushing's syndrome, liver diseases, cancers, and psychiatric disorders by administration of a GRM in such pharmaceutical compositions are also provided.

### CROSS-REFERENCE TO RELATED APPLICATION

This application claims the benefit of, and priority to, U.S. Provisional Application No. 62/504,461, filed May 10, 2017, the entire contents of which is hereby incorporated in its entirety for all purposes.

### BACKGROUND

Steroid molecules, such as steroid hormones, play an important role in bodily functions and in bodily responses to infectious and other diseases, and to the environment. Many steroid molecules are synthesized in the body, or are produced from molecules consumed in the diet. Steroid molecules which act as hormones in the body include estrogen, progesterone, testosterone, and cortisol. Some steroid molecules have medicinal effects. Inhibition of steroid synthesis or metabolism can be useful in the treatment of some disorders.

Cortisol, a steroid molecule, plays an important role in many bodily functions. Cortisol exerts effects by binding to cortisol receptors, which are present in most tissues in the body. However, dysregulation of cortisol may have adverse effects on a subject. For example, Cushing's syndrome, caused by excess levels of cortisol, is characterized by symptoms including elevated blood pressure (hypertension), elevated blood glucose (hyperglycemia), abnormalities in glucose control, requirement for anti-diabetic medication, abnormalities in insulin level, other pre-diabetic symptoms, increased or excessive body weight, increased mid-section perimeter, cushingoid appearance (e.g., a “moon-face” facial appearance), immune suppression, thin skin, acne, depression, hirsutism, abnormal psychiatric symptoms, and other symptoms.

One effective treatment of cortisol dysregulation is to block the binding of cortisol to cortisol receptors, or to block the effect of such cortisol binding to cortisol receptors. Compounds which alter the binding of cortisol to cortisol receptors are termed glucocorticoid receptor modulators (GRMs). GRMs which block or reduce the binding of cortisol to cortisol receptors, or which block or reduce the effects of such binding, are termed glucocorticoid receptor antagonists (GRMs). Mifepristone (11β-(4-dimethylaminophenyl)-17β-hydroxy-17α-(1-propynyl)-estra-4,9-dien-3-one)) is a steroid compound that is a GRM which may act as a GRM. Mifepristone may be provided in pharmaceutical formulations for oral administration.

GRMs may have other chemical forms in addition to steroidal form. Non-steroidal GRMs include, compounds disclosed in, e.g., U.S. Pat. No. 7,576,076; U.S. Pat. No. 7,678,813; U.S. Pat. No. 7,928,237; U.S. Pat. No. 8,461,172; U.S. Pat. No. 8,598,154; U.S. Pat. No. 8,685,973; U.S. Pat. No. 8,859,774; U.S. Pat. No. 8,889,867; U.S. Pat. No. 9,321,736; and U.S. Patent Publication 2015-0148341 (the disclosures of which patents and patent publications are all hereby incorporated by reference in their entireties), among many compounds which differ chemically from mifepristone and also act as glucocorticoid receptor antagonists.

To be useful, a pharmaceutically active compound must be formulated in a suitable pharmaceutical formulation for delivery to a subject suffering from a disease or condition which may respond to treatment by the compound. However, many compounds are difficult to formulate in compositions suitable for safe and effective administration to patients. Some compounds may require excessive amounts of solvents, or may require the use of toxic solvents, in order to be contained in sufficient amounts in a liquid or solid formulation. Some compounds may be unstable, or may be incompatible with pill materials (e.g., may cause degradation or breakdown of capsules in which the compounds are contained), or may separate or precipitate over time, or otherwise become unsuitable for administration.

Mifepristone, a steroidal GRM compound, has been formulated for oral delivery. However, other GRM compounds, including non-steroidal GRM compounds, may have chemical or physical properties which prevent the inclusion of effective amounts in pharmaceutical compositions suitable for administration to patients.

Accordingly, improved formulations which allow for effective amounts of non-steroidal GRMs to be incorporated in pharmaceutical formulations suitable for administration to patients are desired.

### SUMMARY

Many compounds, including many non-steroidal compounds, may be difficult to formulate for use in pharmaceuticals suitable for administration to patients. Applicant provides herein formulations and pharmaceutical compositions suitable for administration to patients in need of treatment by a glucocorticoid receptor modulator (GRM), including a glucocorticoid receptor antagonist (GRA). In embodiments, the GRMs may be, for example, GRMs that increase cortisol levels in subjects to which they have been administered. The GRM may be, e.g., a non-steroidal GRM (which may be, e.g., a non-steroidal GRA). In embodiments, non-steroidal GRMs may include, for example, pyrimidinediones; azadecalins; fused-ring azadecalins; heteroaryl-ketone fused-ring azadecalins; octahydro fused azadecalins, and other compounds. The formulations and pharmaceutical compositions suitable for administration to patients include GRMs as active ingredients in the formulations.

Applicant discloses multiple formulations of GRMs suitable for pharmaceutical use, and provides herein pharmaceutical compositions including GRMs. In embodiments, the formulations may include a GRM and a pharmaceutically acceptable excipient. In embodiments, the formulations contain a GRM and a pharmaceutically acceptable excipient and are suitable for use in pharmaceutical compositions for oral administration of the GRM. In embodiments, the GRM is a non-steroidal GRM. In embodiments, the GRM is a compound disclosed in U.S. Patent Publication 2015-0148341. In an embodiment, the GRM is ((4aR,8aS)-1-(4-fluorophenyl)-6-((2-methyl-2H-1,2,3-triazol-4-yl)sulfonyl)-4,4a,5,6,7,8,8a, 9-octahydro-1H-pyrazolo[3,4-g]isoquinolin-4a-yl)(4-(trifluoromethyl)pyridin-2-yl)methanone (Example 2C of U.S. Patent Publication 2015-0148341, termed “CORT125281”).

In embodiments, the GRM is suitable for use in the treatment of disorders characterized by cortisol excess. In embodiments, the GRM is suitable for use in the treatment of Cushing's syndrome, including Cushing's Disease. In embodiments, the GRM suitable for use in the treatment of Cushing's syndrome, including Cushing's Disease, increases cortisol levels in subjects to which they have been administered, among the effects of administration of the GRM. In embodiments, the GRM is suitable for use in the treatment of neoplastic disorders, including cancer. In embodiments, the GRM is suitable for use in the treatment of liver diseases and disorders. Methods of treating diseases including Cushing's syndrome, cancer, liver diseases, psychiatric disorders, and other diseases and disorders by administration of a GRM in such pharmaceutical compositions are also provided. Such treatments may be administered alone, or in combination with other treatments for such diseases and disorders.

The formulations and methods disclosed herein may be useful in treating patients suffering from a condition amenable to treatment with a GRM. Conditions amenable to treatment with a GRM may include without limitation, for example, Cushing's syndrome, Cushing's Disease, prostate cancer, breast cancer, ovarian cancer, cervical cancer, other hormone-sensitive cancer, other cancer (e.g., pancreatic cancer), liver disease (including a fatty liver disease, cirrhosis, fibrosis, and other liver diseases), depression, dementia, stress disorders (including post-traumatic stress disorder, anxiety, and other stress disorders), substance abuse disorders, and other diseases, disorders, and conditions. For example, the formulations and pharmaceutical compositions disclosed herein, including formulations and pharmaceutical compositions containing CORT125281 as the active ingredient, may be useful for treating prostate cancer and other cancers.

The formulations and pharmaceutical compositions disclosed herein provide advantages including improved formulations including GRMs as active ingredients, and treatments using such improved formulations.

### DETAILED DESCRIPTION

Glucocorticoid receptor modulators (GRMs, including GRAs) have found use in treating diseases and disorders, including, for example, Cushing's syndrome, Cushing's disease, and other disorders which involve disorders of cortisol or other aspects of the hypothalamic pituitary adrenal (HPA) axis. In addition, GRMs may be useful in treating, alone or in combination with other treatments, disorders including, e.g., prostate cancer, breast cancer, ovarian cancer, and other hormone sensitive cancers (see, e.g., U.S. Pat. No. 8,710,035; U.S. Pat. No. 9,149,485; U.S. Pat. No. 9,289,436), liver diseases and conditions (see, e.g., U.S. Patent Application Publication 2016/0106749), and including other disorders (see, e.g., U.S. Pat. No. 6,150,349; U.S. Pat. No. 6,369,046; U.S. Pat. No. 6,620,802; U.S. Pat. No. 6,680,310; U.S. Pat. No. 6,964,953; U.S. Pat. No. 7,163,934; U.S. Pat. No. 7,361,646; U.S. Pat. No. 7,402,578; U.S. Pat. No. 7,884,091; U.S. Pat. No. 8,097,606; U.S. Pat. No. 8,450,379; U.S. Pat. No. 8,476,254; U.S. Pat. No. 8,741,880; U.S. Pat. No. 8,598,149; U.S. Pat. No. 8,921,348). In embodiments, the GRMs may, as one of their effects, increase cortisol levels in subjects to which they have been administered. In embodiments, for example, the formulations and pharmaceutical compositions disclosed herein, including formulations and pharmaceutical compositions containing CORT125281 as the active ingredient, may be useful for treating any of the disorders discussed above or elsewhere in the application.

All patents, patent applications, and patent publications discussed herein, both supra and infra, are hereby incorporated by reference in their entireties for all purposes.

Applicant has surprisingly developed multiple formulations which allow the incorporation of many non-steroidal GRM compounds into pharmaceutical compositions suitable for administration to patients. Applicant has determined that, in embodiments, formulations comprising non-steroidal GRM compounds and including Transcutol, Vitamin E, Kolliphor, Capryol, Gelucire, or Triacetin, or combinations thereof, may provide superior pharmaceutical formulations as compared to prior or alternative formulations. Applicant has determined that, in embodiments, formulations comprising non-steroidal GRM compounds and including Transcutol, Vitamin E, or both, may provide superior pharmaceutical formulations as compared to prior or alternative formulations. Applicant discloses herein formulations which allow the incorporation of many of the non-steroidal GRM compounds into pharmaceutical compositions suitable for administration to patients. In embodiments, the non-steroidal GRM compounds may be non-steroidal GRA compounds. In embodiments, the non-steroidal GRMs may, as one of their effects, increase cortisol levels in subjects to which they have been administered.

In embodiments, Applicant discloses herein formulations containing non-steroidal GRM compounds, including without limitation the following formulations:
a formulation containing a non-steroidal GRM which consists of (% w/w) about 8% to about 14% of said non-steroidal GRM, about 7% to about 10% Transcutol HP, about 33% to about 38% Gelucire, about 29% to about 33% Vitamin E, and about 12% to about 15% PEG400;
a formulation containing a non-steroidal GRM which consists of (% w/w) about 8% to about 14% of said non-steroidal GRM, about 18% to about 22% Transcutol HP, about 28% to about 32% Gelucire, about 25% to about 28% Vitamin E TPGS, and about 8% to about 14% PEG400;
a formulation containing a non-steroidal GRM which consists of (% w/w) about 8% to about 14% of said non-steroidal GRM, about 8% to about 12% Transcutol HP, about 28% to about 32% Kolliphor HS15, and about 47% to about 51% Gelucire 44/14;
a formulation containing a non-steroidal GRM which consists of (% w/w) about 8% to about 14% of said non-steroidal GRM, about 18% to about 22% PEG400, about 38% to about 42% Vitamin E, and about 28% to about 32% Kolliphor HS15;
a formulation containing a non-steroidal GRM which consists of (% w/w) about 8% to about 14% of said non-steroidal GRM, about 33% to about 38% Capryol 90, about 24% to about 29% Tween 20, and about 24% to about 29% Tween 80;
a formulation containing a non-steroidal GRM which consists of (% w/w) about 8% to about 14% of said non-steroidal GRM, about 14% to about 18% medium chain triglycerides, about 37% to about 43% Tween 20, and about 21% to about 26% Kolliphor RH40;
a formulation containing a non-steroidal GRM which consists of (% w/w) about 8% to about 14% of said non-steroidal GRM, about 66% to about 73% Vitamin E, and about 18% to about 22% Transcutol HP;
a formulation containing a non-steroidal GRM which consists of (% w/w) about 7% to about 13% of said non-steroidal GRM, about 7% to about 11% Transcutol, about 52% to about 56% Labrasol, and about 25% to about 29% Kolliphor HS 15;
a formulation containing a non-steroidal GRM which consists of (% w/w) about 8% to about 14% of said non-steroidal GRM, about 7% to about 11% Transcutol HP, about 18% to about 22% Kolliphor RH40, about 48% to about 52% Gelucire 44/14, and about 8% to about 12% Triacetin;
a formulation containing a non-steroidal GRM which consists of (% w/w) about 8% to about 14% of said non-steroidal GRM, about 55% to about 63% Vitamin E TPGS, about 7% to about 11% Transcutol HP, and about 19% to about 23% Triacetin; and
a formulation containing a non-steroidal GRM which consists of (% w/w) about 8% to about 14% of said non-steroidal GRM, about 65% to about 73% Vitamin E TPGS, about 7% to about 11% Transcutol HP, and about 9% to about 13% Triacetin.

In embodiments, Applicant discloses herein single unit dosage forms containing non-steroidal GRM compounds, including without limitation the following single unit dosage forms:
a single unit dosage form consisting of a capsule containing a uniform admixture of between about 10 milligrams (mg) and about 70 mg of a non-steroidal GRM and pharmaceutically acceptable excipients, wherein said capsule has a size selected from size 5, size 4, size 3, size 2, size 1, size 0, size 00, and size 000, and wherein said uniform admixture is a formulation as disclosed herein, including, e.g., a formulation disclosed above; and
a single unit dosage form consisting of a capsule containing a uniform admixture of between about 20 milligrams (mg) and about 50 mg of a non-steroidal GRM and pharmaceutically acceptable excipients, wherein said capsule has a size selected from size 5, size 4, size 3, size 2, size 1, size 0, size 00, and size 000, and wherein said uniform admixture is a formulation as disclosed herein, including, e.g., a formulation disclosed above. In embodiments, the contents of a single unit dosage form as disclosed herein, including as disclosed above, weighs between about 300 milligrams (mg) and about 600 mg.

In embodiments, Applicant discloses herein methods of treating conditions amenable to treatment with a GRM, including without limitation the following methods:
a method of treating a condition amenable to treatment with a GRM comprising administering a non-steroidal GRM in a single unit dosage form, wherein said single unit dosage form contains a uniform admixture of said non-steroidal GRM and pharmaceutical excipients, and wherein said uniform admixture is a formulation as disclosed herein. In embodiments of the methods disclosed herein, the single unit dosage form consists of a capsule containing between about 10 milligrams (mg) and about 70 mg of said non-steroidal GRM, wherein said capsule has a size selected from size 5, size 4, size 3, size 2, size 1, size 0, size 00, and size 000.

In embodiments of the methods disclosed herein, the single unit dosage form consists of a capsule containing a uniform admixture of between about 20 milligrams (mg) and about 50 mg of said non-steroidal GRM, wherein said capsule has a size selected from size 5, size 4, size 3, size 2, size 1, size 0, size 00, and size 000.

In embodiments of the methods disclosed herein, the condition amenable to treatment with a GRM is selected from Cushing's syndrome, Cushing's Disease, prostate cancer, breast cancer, ovarian cancer, other hormone-sensitive cancer, other cancer, liver disease (including a fatty liver disease, fibrosis, cirrhosis, and other liver diseases), a psychiatric disorder (including, e.g., depression), dementia, stress disorders (including post-traumatic stress disorder, anxiety, and other stress disorders), and substance abuse disorders. In embodiments of the methods disclosed herein, the condition amenable to treatment with a GRM is a hormone-sensitive cancer selected from the group consisting of prostate cancer, breast cancer, and ovarian cancer. In embodiments of the methods disclosed herein, the condition amenable to treatment with a GRM is Cushing's Syndrome.

In embodiments, Applicant discloses herein formulations containing non-steroidal GRMs, and discloses GRM-containing pharmaceutical compositions suitable for administration to patients, of octahydro fused azadecalin GRM compounds having the following formula:

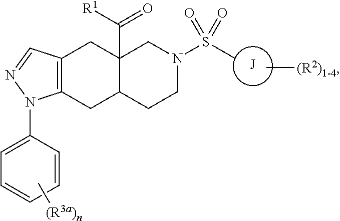

wherein R¹ is a heteroaryl ring having from 5 to 6 ring members and from 1 to 4 heteroatoms each independently selected from the group consisting of N, O and S, optionally substituted with 1-4 groups each independently selected from R^{1a}; each R^{1a} is independently selected from the group consisting of hydrogen, C₁₋₆ alkyl, halogen, C₁₋₆ haloalkyl, C₁₋₆ alkoxy, C₁₋₆ haloalkoxy, N-oxide, and C₃₋₈ cycloalkyl; ring J is selected from the group consisting of an aryl ring and a heteroaryl ring having from 5 to 6 ring members and from 1 to 4 heteroatoms each independently selected from the group consisting of N, O and S; each R² is independently selected from the group consisting of hydrogen, C₁₋₆ alkyl, halogen, C₁₋₆ haloalkyl, C₁₋₆ alkoxy, C₁₋₆ haloalkoxy, C₁₋₆ alkyl-C₁₋₆ alkoxy, CN, OH, NR^{2a}R^{2b}, C(O)R^{2a}, C(O)OR^{2a}, C(O)NR^{2a}R^{2b}, SR^{2a}, S(O)R^{2a}, S(O)₂R^{2a}, C₃₋₈ cycloalkyl, and C₃₋₈ heterocycloalkyl having from 1 to 3 heteroatoms each independently selected from the group consisting of N, O and S; alternatively, two R² groups on adjacent ring atoms are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2c} groups; R^{2a}, R^{2b} and R^{2c} are each independently selected from the group consisting of hydrogen and C₁₋₆ alkyl; each R^{3a} is independently halogen; and subscript n is an integer from 0 to 3, or salts and isomers thereof. Examples of such compounds are disclosed in U.S. Patent Application Publication 2015-0148341, hereby incorporated by reference in its entirety.

In particular embodiments, Applicant discloses herein formulations containing non-steroidal GRMs. In embodiments, Applicant discloses herein formulations which allow the incorporation of many of the non-steroidal GRM compounds disclosed in U.S. Patent Publication 2015-0148341, hereby incorporated by reference herein in its entirety, into pharmaceutical compositions suitable for administration to patients. In a particular embodiment, Applicant discloses GRM-containing pharmaceutical compositions suitable for administration to patients, of the non-steroidal GRM (termed CORT125281) ((4aR,8aS)-1-(4-fluorophenyl)-6-((2-methyl-2H-1,2,3-triazol-4-yl)sulfonyl)-4,4a,5,6,7,8,8a,9-octahydro-1H-pyrazolo[3,4-g]isoquinolin-4a-yl)(4-(trifluoromethyl)pyridin-2-yl)methanone (termed “CORT125281”), which has the structure:

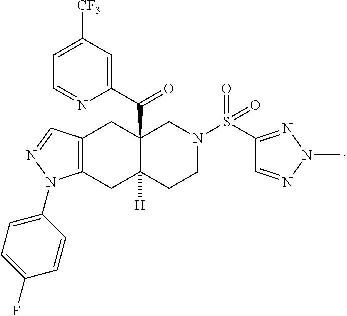 [SMILES: Cn1ncc(S(=O)(=O)N2CC[C@H]3Cc4c(cnn4-c4ccc(F)cc4)C[C@]3(C(=O)c3cc(C(F)(F)F)ccn3)C2)n1]

CORT125281 is disclosed in Example 2C of U.S. Patent Application Publication 2015-0148341. Further non-steroidal GRMs suitable for use in GRM-containing pharmaceutical compositions as disclosed herein include, e.g., the compound disclosed in Example 2A of U.S. Patent Application Publication 2015-0148341; the compound disclosed in Example 2B of U.S. Patent Application Publication 2015-0148341; the compound disclosed in Example 2D of U.S. Patent Application Publication 2015-0148341; the compound disclosed in Example 2E of U.S. Patent Application Publication 2015-0148341; the compound disclosed in Example 2N of U.S. Patent Application Publication 2015-0148341; the compound disclosed in Example 2P of U.S. Patent Application Publication 2015-0148341; the compound disclosed in Example 2R of U.S. Patent Application Publication 2015-0148341; the compound disclosed in Example 2AD of U.S. Patent Application Publication 2015-0148341; the compound disclosed in Example 2AJ of U.S. Patent Application Publication 2015-0148341; and other non-steroidal GRM compounds.

Accordingly, Applicant discloses herein formulations and pharmaceutical compositions containing a GRM. These formulations may be used to provide pharmaceutical compositions for administration to subjects suffering from any disorder or condition which may respond to treatment using a GRM, such as a non-steroidal GRM. Such formulations and pharmaceutical compositions containing a non-steroidal GRM may be used to treat patients suffering from diseases and conditions including, without limitation, Cushing's syndrome, Cushing's Disease, prostate cancer, breast cancer, ovarian cancer, cervical cancer, other hormone-sensitive cancer, pancreatic cancer, other cancer, liver disease (including a fatty liver disease, fibrosis, cirrhosis, and other liver diseases), obesity, diabetes, cardiovascular disease, hypertension, Syndrome X, depression, anxiety, glaucoma, human immunodeficiency virus (HIV) or acquired immunodeficiency syndrome (AIDS), neurodegeneration, mild cognitive impairment, dementia, Alzheimer's disease, Parkinson's disease, Huntington's disease, cognition enhancement, Addison's Disease, osteoporosis, frailty, muscle frailty, inflammatory diseases, osteoarthritis, rheumatoid arthritis, asthma and rhinitis, adrenal function-related ailments, viral infection, immunodeficiency, immunomodulation, autoimmune diseases, allergies, wound healing, compulsive behavior, multi-drug resistance, psychosis, anorexia, cachexia, stress disorders (including post-traumatic stress disorders, and other stress disorders), substance abuse disorders, addiction, post-surgical bone fracture, medical catabolism, depression (including major psychotic depression), psychosis, hyperglycemia, central serous retinopathy, alcohol dependence, stress disorders, antipsychotic induced weight gain, delirium, cognitive impairment in depressed patients, cognitive deterioration in individuals with Down's syndrome, psychosis associated with interferon-alpha therapy, chronic pain, pain associated with gastroesophageal reflux disease, postpartum psychosis, postpartum depression, neurological disorders in premature infants, migraine headaches, and other diseases, disorders, and conditions.

Thus, Applicant discloses herein surprising and useful formulations which allow incorporation of useful amounts of GRM in forms suitable for use in pharmaceutical compositions, and provides pharmaceutical compositions including useful amounts of non-steroidal GRMs. In embodiments, the formulations and pharmaceutical compositions include formulations and pharmaceutical compositions containing the GRM compound termed CORT 125281.

Applicant provides definitions of some terms used in the present disclosure.

### Definitions

The abbreviations used herein have their conventional meaning within the chemical and biological arts.

“Patient”, “subject”, “subject in need” and the like refer to a person having, or suspected of having, a disease or condition which may be treated by administration of a therapeutic drug.

As used herein, the term “Cushing's syndrome” refers to an array of symptoms caused by excess cortisol. Such symptoms include, for example, elevated blood pressure (hypertension), elevated blood glucose (hyperglycemia), increased weight (typically in the mid-section, and in the face causing a characteristic “moon-face”), immune suppression, thin skin, acne, depression, hirsutism, and other symptoms. The excess cortisol that leads to Cushing's syndrome may be due to exogenous cortisol (or cortisol analog such as prednisone, cortisone, dexamethasone, or other cortisol analog or cortisol mimetic); may be due to a pituitary tumor; may be due to an adrenal tumor or other adrenal disease; may be due to a tumor not located in or near to the pituitary or an adrenal gland; or may be due to another cause.

As used herein, “Cushing's Disease” refers to pituitary-dependent Cushing's syndrome, e.g., excess cortisol caused by pituitary abnormality (typically a pituitary tumor). Cushing's Disease is thus a disease that is a particular type of Cushing's syndrome. The term Cushing's syndrome thus includes Cushing's Disease as well as other disorders characterized by cortisol excess.

As used herein, a “patient suffering from Cushing's syndrome” refers to any patient suffering from Cushing's syndrome, including endogenous Cushing's syndrome; Cushing's Disease; or a condition associated with Cushing's syndrome. A condition associated with Cushing's syndrome may be, without limitation, a condition associated with endogenous Cushing's syndrome; hyperglycemia secondary to hypercortisolism; a condition of hypercortisolism in an endogenous Cushing's syndrome patient, said patient having type 2 diabetes mellitus or glucose intolerance; a condition of hyperglycemia secondary to hypercortisolism in an endogenous Cushing's syndrome patient, said patient having type 2 diabetes mellitus or glucose intolerance and having failed surgery; hyperglycemia secondary to hypercortisolism in an endogenous Cushing's syndrome patient, said patient having type 2 diabetes mellitus or glucose intolerance and having failed surgery or who is not a candidate for surgery; and other conditions associated with Cushing's syndrome.

As used herein, the term “administration” refers to the delivery of a drug or other therapeutic into the body of a subject in need of treatment by the drug or therapeutic, effective to achieve a therapeutic effect. Administration may be by any suitable route of administration, including, for example, oral administration; intravenous administration; subcutaneous administration; parenteral administration; intra-arterial administration; nasal administration; topical administration; and other routes of administration.

The term “which may respond to treatment” refers to a disorder or condition, which may be successfully treated by, or the symptoms of which may be improved or eliminated by, administration of a compound such as, e.g., a GRM compound. Thus, a disease or disorder which may respond to treatment by a compound is one in which the disease, disorder, or symptoms thereof, may be ameliorated or eradicated by administration of the compound. A subject having a disorder or condition which may benefit from the treatment may thus experience relief from, abatement of, or cure of the disease or condition.

“Treat”, “treating” and “treatment” refers to providing a drug or other therapeutic agent to a patient. Such treatments are intended to, and typically result in reduction of symptoms, or amelioration of symptoms, or abatement of symptoms, or abolition of symptoms, of the disease or disorder to be treated. Such treatments are intended to, and typically result in, an indication of success in the treatment or amelioration of a pathology or condition. Indicia of success include, e.g., any objective or subjective parameter such as abatement; remission; diminishing of symptoms or making the pathology or condition more tolerable to the patient; slowing in the rate of degeneration or decline; making the final point of degeneration less debilitating; or improving a patient's physical or mental well-being. The treatment or amelioration of symptoms can be based on objective or subjective parameters; including the results of a physical examination; histopathological examination (e.g., analysis of biopsied tissue); laboratory analysis of urine, saliva, tissue samples, serum, plasma, or blood; or imaging.

As used herein, “treating a patient who is suffering from Cushing's syndrome”, or treating a subject who is suffering from Cushing's syndrome”, or similar phrases refer to, without limitation, treating a patient suffering from Cushing's syndrome, including endogenous Cushing's syndrome; treating a patient suffering from Cushing's Disease; or treating a patient suffering from a condition associated with Cushing's syndrome. Cushing's syndrome, Cushing's Disease, and a condition associated with Cushing's syndrome are discussed above. For example, treating a patient who is suffering from Cushing's syndrome may include administering a GRM to control hyperglycemia secondary to hypercortisolism in adult patients with endogenous Cushing's syndrome who have type 2 diabetes mellitus or glucose intolerance and have failed surgery or are not candidates for surgery.

As used herein, “treating a patient who is suffering from cancer”, or a similar phrase, refers without limitation to treating a patient suffering from any cancer, including without limitation prostate cancer, breast cancer, ovarian cancer, cervical cancer, pancreatic cancer, and other cancers (e.g., see below).

As used herein, or “treating a patient who is suffering from a hormone-sensitive cancer” refers without limitation to treating a patient suffering from a cancer that is typically hormone sensitive (e.g., typically sensitive to estrogen, or to testosterone, or other hormone), and includes treating cancer that may have lost its sensitivity to a hormone during the course of the disease. Hormone-sensitive cancers include, without limitation, prostate cancer, testicular cancer, breast cancer, ovarian cancer, cervical cancer, endometrial cancer, thyroid cancer, osteosarcoma, and other cancers.

As used herein, the terms “percent” and “%” refer to a percentage taken by comparing a first value to a second value, and multiplying the resulting decimal fraction by 100. As used herein, the first value may be the weight of an ingredient in a formulation containing multiple ingredients, and the second value may be the weight of all ingredients (i.e., the total weight of the formulation). Thus, for example, where the weight of a GRM dose in a pharmaceutical formulation containing that GRM is 100 milligrams (mg), and the total weight of all the ingredients in the formulation is 400 mg, then the GRM makes up 25% of the formulation.

As used herein, the terms “weight percent”, “weight %”, “(% w/w)” and the like refer to the percentage of an ingredient of a composition with respect to the total weight of a composition containing at least two ingredients.

As used herein, the phrase “between about x % and about y %” (where x and y may be any number) is used inclusively, so that the range include the number values stated and all values between those minimum and maximum values.

As used herein, the terms “effective amount,” “amounts effective,” therapeutic amount”, and “therapeutically effective amount” refer to an amount or amounts of one or more pharmacological agents effective to treat, eliminate, or mitigate at least one symptom of the disease being treated. In some cases, “effective amount,” “amounts effective,” “therapeutic amount”, and “therapeutically effective amount” can refer to an amount of a functional agent or of a pharmaceutical composition useful for exhibiting a detectable therapeutic or inhibitory effect. The effect can be detected by any assay method known in the art.

As used herein, the terms “pharmaceutical composition” and “formulation” refer to compositions suitable for administration to a patient for treatment of a medical condition or for amelioration of symptoms of a medical condition. A pharmaceutical composition as disclosed herein includes an active ingredient (e.g., a GRM, such as, e.g., CORT125281 and a pharmaceutically acceptable excipient. In embodiments, a pharmaceutical composition includes one or more active ingredients and one or more pharmaceutically acceptable excipients.

As used herein, the terms “sustained release,” “slow release,” “long acting,” “prolonged release,” and the like refer to a pharmaceutical composition or formulation containing at least one active ingredient (e.g., a GRM) formulated to maintain a therapeutic concentration of active ingredient(s) in a patient for a longer period of time in comparison to formulations that are not designed for such sustained release. In some cases, the sustained release formulation maintains therapeutic concentration of one or more active ingredient(s) for, or for at least, one hour; two hours; three hours; four hours; five hours; eight hours; ten hours; twelve hours; twenty four hours; two days; four days; or more up to and including, for example, one week, two weeks, three weeks, four weeks, five weeks, or six weeks). In some cases, the sustained release formulation is administered to a patient every day; every two days; every three days; every four days; up to and including, e.g., every one, two, three, four, five, or six weeks.

In embodiments, formulations containing a GRM disclosed herein may be suitable for use in the manufacture of pharmaceutical compositions for administration to patients, and pharmaceutical compositions containing a GRM may be pharmaceutical compositions suitable for administration of the GRM to patients.

In embodiments, formulations containing a GRM disclosed herein may be suitable for use in the manufacture of pharmaceutical compositions for oral administration.

In embodiments, pharmaceutical compositions containing a GRM may be pharmaceutical compositions suitable for oral administration of the GRM.

In embodiments, formulations and pharmaceutical compositions containing a GRM, useful for pharmaceutical compositions for administration including for oral administration, may include excipients.

As used herein, the term “excipient” is used to refer to any compound or material which is included in a formulation or in a pharmaceutical composition other than the active ingredient (or active ingredients where more than one compound may have the desired pharmaceutical activity). An excipient may serve as, e.g., a solvent, solubilizer or solubility enhancer; an emulsifier; a bulking agent; a stabilizer; a diluent; a surfactant; a preservative; a colorant; a flavor; a filler; a lubricant; or other agent which may serve other functions.

As used herein, the terms “pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, sodium chloride (NaCl), normal saline solutions, lactated Ringer's, normal sucrose, normal glucose, binders, fillers, disintegrants, lubricants, coatings, sweeteners, flavors and colors, and the like. One of skill in the art will recognize that other pharmaceutical excipients are useful in the present invention.

Solvents, solubilizers, and solubility enhancers used as excipients in formulations and pharmaceutical compositions as disclosed herein may include polyethylene glycols (PEG), ethanol, propylene glycol, glycerin, N-methyl-2-pyrrolidone, dimethylacetamide, dimethylsulfoxide, and other water-soluble organic solvents. In embodiments, formulations and pharmaceutical compositions as disclosed herein containing a GRM may include diethylene glycol monoethyl ether (sold under the names Transcutol®, Carbitol®, dioxitol, and other names); Transcutol®, e.g., Transcutol® HP, is available from Gattefosse, Saint-Priest, Lyon, France.

Polyethylene glycols are used as excipients. Polyethylene glycols of many forms, and derivatives thereof, are useful as solvents, surfactants, and in other ways in the formulations and pharmaceutical compositions disclosed herein. For example, PEG may have mean molecular weight of 300 kD or 400 kD (e.g., polyethylene glycol 300, polyethylene glycol 400); may be used in succinate form as polyethylene glycol (PEG) succinates (e.g., polyethylene glycol 1000 succinate) and in the form of mono- and di-fatty acid esters of PEG (e.g., PEG 300, 400, and 1750 mono- and di-fatty acid esters); and in other forms. Polyethylene glycols of a wide range of molecular weights (e.g., PEG300 which has an average molecular weight of about 300 grams per mole, and PEG400 which has an average molecular weight of about 400 grams per mole) are available, e.g., from SIGMA-Aldrich, St. Louis, Mo., USA.

For example, polysorbate (polyoxyethylene sorbitan monolaureate) surfactants and detergents (such as, e.g., Tween® 20, Tween® 80, and others, where numbers such as “20” and “80” indicate the numbers of repeating polyethylene glycol units in the polymers) are used as excipients in formulating pharmaceuticals, and aid in the emulsification and/or solubilization of active compounds in addition to their actions as surfactants. Such nonionic detergents and surfactants are available, e.g., from SIGMA-Aldrich, St. Louis, Mo., USA).

Excipients used in formulations and pharmaceutical compositions as disclosed herein may include lipids and phospholipids, including naturally occurring lipid compositions such as, e.g., castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil and palm seed oil). Excipients used in formulations and pharmaceutical compositions as disclosed herein may also include phospholipids such as, e.g., phosphatidylcholine, distearoylphosphatidylglycerol L-α-dimyristoylphosphatidylcholine, L-α-dimyristoylphosphatidylglycerol, and other phospholipids. Lipids and phospholipids may be used, e.g., as emulsifiers, bulking agents, fillers, lubricants, and for other purposes.

In embodiments, formulations and pharmaceutical compositions as disclosed herein containing a GRM may include triglycerides, including medium chain triglycerides. Medium chain triglycerides are triglycerides with fatty acid chain lengths of between about 6 to about 12 carbons long. Triglycerides may include, e.g., 1,2,3-triacetoxypropane (also known as triacetin or as glycerin triacetate). Triacetin is available, e.g., from SIGMA-Aldrich, St. Louis, Mo., USA. Triglyceride excipients such as, e.g., triacetin, may serve, e.g., as solvents, as plasticizers, and as humectants.

Excipients used in formulations and pharmaceutical compositions as disclosed herein may include organic materials (which may be liquid or may be semi-solid at room temperature) such as, e.g., beeswax, d-α-tocopherol (also termed vitamin E, or vitE), oleic acid, gum Arabic, lanolin, starch, syrup, honey, and medium-chain mono- and diglycerides. Such organic materials may be used, e.g., as bulking agents, fillers, lubricants, and for other purposes.

Excipients used in formulations and pharmaceutical compositions as disclosed herein may include various cyclodextrins such as, e.g., α-cyclodextrin, β-cyclodextrin, hydroxypropyl-β-cyclodextrin, sulfobutylether-β-cyclodextrin, and others. Cyclodextrins may be used, e.g., as emulsifiers, solubilizers, and for other purposes.

Surfactants, including non-ionic surfactants, are used as excipients. Surfactant excipients used in formulations and pharmaceutical compositions as disclosed herein may include non-ionic surfactants such as sorbitan monooleate; polysorbates (of many sizes, including e.g., polysorbate 20, polysorbate 80, also known as Tween® 20 and Tween® 80 as discussed above); and other non-ionic surfactants including, e.g., those sold as Cremophor® EL, Cremophor® RH 40, Cremophor® RH 60, d-α-tocopherol, Solutol HS 15, poloxamer 407, Labrafil® M-1944CS (Gattefosse), Labrafil M-2125CS (Gattefosse), Labrasol® (Gattefosse, Saint-Priest, Lyon, France), Softigen® 767), and mono- and di-fatty acid esters of PEG.

In embodiments, formulations and pharmaceutical compositions as disclosed herein containing a GRM may include Gelucire (e.g., Gelucire® 44/14, available from Gattefosse, Saint-Priest, Lyon, France) as a surfactant.

In embodiments, formulations and pharmaceutical compositions as disclosed herein containing a GRM may include a “Kolliphor” as a surfactant. Kolliphor® EL (also known as cremophor; see above) is a polyethoxylated castor oil used as a non-ionic oil-in-water emulsifier and/or solubilizer. Kolliphor P188 is a poloxamer. Kolliphor® HS 15 (polyethylene glycol (15)-hydroxystearate is a solubilizer/emulsifier. Kolliphor® RH40 is Macrogolglycerol hydroxystearate These materials are available from SIGMA-Aldrich, St. Louis, Mo., USA.

In embodiments, formulations and pharmaceutical compositions as disclosed herein containing a GRM may include Propylene Glycol Monocaprylate (sold, e.g., as Capryol™ 90 by Gattefosse, Saint-Priest, Lyon, France).

In embodiments, formulations and pharmaceutical compositions as disclosed herein containing a GRM may include Caprylocaproyl polyoxylglycerides such as Labrasol® (sold by Gattefosse, Saint-Priest, Lyon, France; see above).

In embodiments, formulations and pharmaceutical compositions as disclosed herein containing a GRM may include Vitamin E (e.g., α-tocopherol and other tocopherols). Vitamin E is available, e.g., from SIGMA-Aldrich, St. Louis, Mo., USA. Vitamin E may serve, e.g., as a solubilizer; as an emulsifier; and as an antioxidant.

In embodiments, formulations and pharmaceutical compositions as disclosed herein containing a GRM may include α-tocopherol polyethylene glycol succinate (Vitamin E TPGS). Vitamin E TPGS is available, e.g., from SIGMA-Aldrich, St. Louis, Mo., USA.

Further excipients and materials useful in formulations and in the manufacture of pharmaceuticals for administration, including pharmaceutical for oral administration, include sterile water for irrigation; ethanol; gelatin (e.g., edible gelatin, having a jelly strength measured in “bloom” units, such as Gelatin 220 Bloom); coatings such as hydroxypropyl methylcellulose (HPMC, “hypromellose”, e.g., Pharmacoat® 603 and Pharmacoat® 615) and polyvinyl alcohol coating materials; and other materials.

Pharmaceutical compositions may include capsules to contain pharmaceutical ingredients. In embodiments, such capsules are suitable for administration to patients. Examples of capsules include capsule shell types including hard gelatin capsules (e.g., Coninsnap®, available from Capsugel, Morristown, N.J., USA); and hydroxypropyl methylcellulose capsules (e.g., Vcaps Plus®, available from Capsugel, Morristown, N.J., USA).

### Compounds

Description of GRM compounds, such as non-steroidal GRM compounds, useful in the formulations and pharmaceutical compositions disclosed herein, including compounds suitable for oral administration, are described in accordance with principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, or physiological conditions.

Where substituent groups are specified by their conventional chemical formulae, written from left to right, they equally encompass the chemically identical substituents that would result from writing the structure from right to left, e.g., —CH₂O— is equivalent to —OCH₂—.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc.

“Alkoxy” refers to an alkyl group having an oxygen atom that connects the alkyl group to the point of attachment: alkyl-O—. As for the alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc.

“Halogen” refers to fluorine, chlorine, bromine and iodine.

“Haloalkyl” refers to alkyl, as defined above, where some or all of the hydrogen atoms are replaced with halogen atoms. As for the alkyl group, haloalkyl groups can have any suitable number of carbon atoms, such as C₁₋₆. For example, haloalkyl includes trifluoromethyl, fluoromethyl, etc. In some instances, the term “perfluoro” can be used to define a compound or radical where all the hydrogens are replaced with fluorine. For example, perfluoromethane includes 1,1,1-trifluoromethyl.

“Haloalkoxy” refers to an alkoxy group where some or all of the hydrogen atoms are substituted with halogen atoms. As for the alkyl group, haloalkoxy groups can have any suitable number of carbon atoms, such as C₁₋₆. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc.

“Cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl.

“Heterocycloalkyl” refers to a saturated ring system having from 3 to 12 ring members and from 1 to 4 heteroatoms of N, O and S. Additional heteroatoms can also be useful, including, but not limited to, B, Al, Si and P. The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxalidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline.

When heterocycloalkyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxazolidine, thiazolidine, isothiazolidine, morpholine, thiomorpholine, dioxane and dithiane. Heterocycloalkyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

“Aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

“Heteroaryl” refers to a monocyclic or fused bicyclic or tricyclic aromatic ring assembly containing 5 to 16 ring atoms, where from 1 to 5 of the ring atoms are a heteroatom such as N, O or S. Additional heteroatoms can also be useful, including, but not limited to, B, Al, Si and P. The heteroatoms can also be oxidized, such as, but not limited to, N-oxide, —S(O)— and —S(O)₂—. Heteroaryl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring heteroatoms including N, O or S, such as pyrrole, pyridine, imidazole, pyrazole, pyrazine, pyrimidine, pyridazine, thiophene, furan, thiazole, isothiazole, oxazole, and isoxazole.

Some heteroaryl groups include from 5 to 10 ring members and only nitrogen heteroatoms, such as pyrrole, pyridine, imidazole, pyrazole, triazole, pyrazine, pyrimidine, pyridazine, triazine (1,2,3-, 1,2,4- and 1,3,5-isomers), indole, isoindole, quinoline, isoquinoline, quinoxaline, quinazoline, phthalazine, and cinnoline. Other heteroaryl groups include from 5 to 10 ring members and only oxygen heteroatoms, such as furan and benzofuran. Some other heteroaryl groups include from 5 to 10 ring members and only sulfur heteroatoms, such as thiophene and benzothiophene. Still other heteroaryl groups include from 5 to 10 ring members and at least two heteroatoms, such as imidazole, pyrazole, triazole, pyrazine, pyrimidine, pyridazine, triazine (1,2,3-, 1,2,4- and 1,3,5-isomers), thiazole, isothiazole, oxazole, isoxazole, quinoxaline, quinazoline, phthalazine, and cinnoline.

“Heteroatoms” refers to O, S or N.

“Salt” refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

As used herein, the terms “steroid” and “steroids”, and the phrase “steroidal backbone” in the context of glucocorticoid receptor antagonists containing such refers to glucocorticoid receptor antagonists that contain modifications of the basic structure of cortisol, an endogenous steroidal glucocorticoid receptor ligand. The basic structure of a steroidal backbone is provided as Formula I:

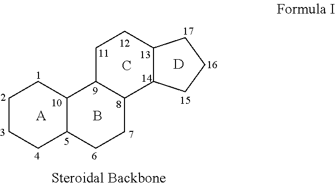

The two most commonly known classes of structural modifications of the cortisol steroid backbone to create glucocorticoid antagonists include modifications of the 11-β hydroxy group and modification of the 17-β side chain (See, e. g., Lefebvre (1989) J. Steroid Biochem. 33: 557-563).

The term “cortisol” refers to the naturally occurring glucocorticoid hormone (also

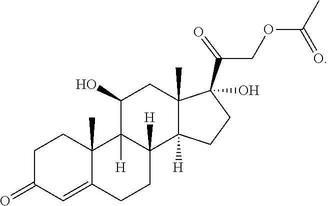

known as hydrocortisone) having the structure:

As used herein, the term glucocorticoid receptor (GR) refers to a receptor that binds a glucocorticoid, such as cortisol, dexamethasone, or other molecules. A glucocorticoid receptor, also known as a corticosteroid receptor or as a type II glucocorticoid receptor (GR II), and in humans, as a cortisol receptor, is activated by cortisol in humans (or, e.g., by corticosterone (“cortisone”) in some other animals, such as rats and mice). The human cortisol receptor (GR II receptor, Genbank: P04150) specifically binds to cortisol and/or cortisol analogs (e.g. dexamethasone). The term includes isoforms of GR II, recombinant GRII, and mutated GRII.

As used herein, the term glucocorticoid receptor modulator (GRM) refers to an agent that affects the action of a glucocorticoid receptor (GR). Such modulation may include activation (agonist action), partial activation (partial agonist action), inhibition (reduction in activation of the receptor under conditions where it would otherwise be activated, such as in the presence of cortisol), and blockade (complete or near complete suppression of activation of the receptor under conditions where it would otherwise be activated, such as in the presence of cortisol). GRMs may affect the activity of a GR by increasing or by decreasing the activity of the GR. GRMs include steroids, and, in embodiments, include non-steroidal compounds, including, for example: pyrimidinediones; azadecalins; fused-ring azadecalins; heteroaryl-ketone fused-ring azadecalins; octahydro fused azadecalins; and other compounds.

As used herein, the terms “glucocorticoid agonist”, “glucocorticoid receptor agonist”, “glucocorticoid receptor type II agonist”, and “GRII agonist” refer to a compound or agent which may bind to and activate a cortisol receptor. Such agents include, for example, cortisol, dexamethosone, prednisone, and other compounds and agents which bind to and activate a GRII.

In embodiments, a glucocorticoid receptor modulator (GRM) is a glucocorticoid receptor antagonist (GRA). In embodiments, the GRM is an antagonist of a glucocorticoid type II (GRII) receptor. In embodiments, the GRM binds preferentially to a GRII receptor as compared to its binding to a glucocorticoid type I (GRI) receptor. In embodiments, the GRM reduces the activation of a GRII receptor. In embodiments, the GRM reduces the activity of a GRII receptor. In embodiments, the GRM is CORT125281. In embodiments, the GRM is a selective inhibitor of the glucocorticoid receptor. In embodiments, the GRM is a specific inhibitor of the glucocorticoid receptor. A “specific glucocorticoid receptor modulator” refers to any composition or compound which inhibits any biological response associated with the binding of a GR to an agonist. By “specific,” we intend the drug to preferentially bind to the GR rather than another nuclear receptors, such as mineralocorticoid receptor (MR) or progesterone receptor (PR). In embodiments, the GRM may bind to a progesterone receptor (PR); a specific GRM binds to a glucocorticoid receptor with higher affinity than it binds to PR. In embodiments, the specific GRM may only poorly bind to PR, or may not measurably bind to PR.

As used herein, the terms “glucocorticoid antagonist”, “glucocorticoid receptor antagonist”, “glucocorticoid antagonist”, “glucocorticoid receptor type II antagonist”, “GRII antagonist”, and “GRA” refer to compounds that inhibit the action of a cortisol receptor; such inhibition may include interfering with the binding of a glucocorticoid agonist such as cortisol, dexamethosone, or other compound or agent which may bind to and activate a cortisol receptor. Thus, the term “glucocorticoid receptor antagonist” (GRA) refers to any glucocorticoid receptor modulator (GRM) which partially or completely inhibits (antagonizes) a) the binding of a glucocorticoid receptor (GR) agonist, such as cortisol, or cortisol analogs, synthetic or natural, to a GR, or b) which partially or completely inhibits (antagonizes) the effect of such binding (e.g., reduced GR activity). Inhibition constants (Kᵢ) for GRAs against the human cortisol receptor may be between about 0.0001 nM and about 1,000 nM; preferably may be between about 0.0005 nM and about 10 nM, and most preferably between about 0.001 nM and about 1 nM.

A “specific glucocorticoid receptor antagonist” refers to any composition or compound which inhibits any biological response associated with the binding of a GR to an agonist. By “specific,” we intend the drug to preferentially bind to the GR rather than another nuclear receptors, such as mineralocorticoid receptor (MR) or progesterone receptor (PR).

By “specific,” the drug preferentially binds to the GR rather than other nuclear receptors, such as mineralocorticoid receptor (MR), androgen receptor (AR), or progesterone receptor (PR). It is preferred that the specific GRM (e.g., GRA) bind GR with an affinity that is ten-fold greater ( 1/10^{th} the K_{d} value) than its affinity to the MR, AR, or PR. In a more preferred embodiment, the specific GRM (e.g., GRA) binds GR with an affinity that is one hundred-fold greater ( 1/100^{th} the K_{d} value) than its affinity to the MR, AR, or PR.

As used herein, a “steroidal glucocorticoid receptor modulator” means a molecule including a steroid backbone structure which modulates (e.g., antagonizes) the binding of cortisol, corticosterone, or dexamethasone to a glucocorticoid receptor, or which modulates (e.g., antagonizes) the activation of a glucocorticoid receptor by cortisol, corticosterone, or dexamethasone. Examples of steroidal glucocorticoid receptor modulators include mifepristone (11β-(4-dimethylaminophenyl)-17β-hydroxy-17α-(1-propynyl)-estra-4,9-dien-3-one), also referred to as RU486, or as RU38.486, or as 17-beta-hydroxy-11-beta-(4-dimethyl-aminophenyl)-17-alpha-(1-propynyl)-estra-4,9-dien-3-one), monodemethylated mifepristone, didemethylated mifepristone, 17-α-[3′-hydroxy-propynyl]mifepristone, ulipristal (CDB-2914), CDB-3877, CDB-3963, CDB-3236, CDB-4183, cortexolone, dexamethasone-oxetanone, 19-nordeoxycorticosterone, 19-norprogesterone, cortisol-21-mesylate; dexamethasone-21-mesylate, 11(-(4-dimethylaminoethoxyphenyl)-17(-propynyl-17(-hydroxy-4,9-estradien-3one, and 17(-hydroxy-17(-19-(4-methylphenyl)androsta-4,9(11)-dien-3-one.

As used herein, the phrase “non-steroidal backbone” in the context of GRMs containing such refers to GRMs that do not share structural homology to, or are not modifications of, cortisol. Such compounds include, for example, small molecules, synthetic mimetics and analogs of proteins, including partially peptidic, pseudopeptidic and non-peptidic molecular entities.

As used herein, a “non-steroidal glucocorticoid receptor modulator” means a molecule lacking a steroid backbone structure which modulates (e.g., antagonizes) the binding of cortisol, corticosterone, or dexamethasone to a glucocorticoid receptor, or which modulates (e.g., antagonizes) the activation of a glucocorticoid receptor by cortisol, corticosterone, or dexamethasone.

In some embodiments, the GRM is a non-steroidal compound, i.e. a “non-steroidal glucocorticoid receptor modulator”. In embodiments, non-steroidal GRM compounds include compounds having a cyclohexyl-pyrimidine backbone; non-steroidal GRM compounds having a fused azadecalin backbone; non-steroidal GRM compounds having a heteroaryl ketone fused azadecalin backbone; and non-steroidal GRM compounds having an octahydro fused azadecalin backbone. Exemplary non-steroidal glucocorticoid receptor antagonists include those described in U.S. Pat. Nos. 8,685,973; 7,928,237; 8,461,172; and 8,859,774, and include those described in U.S. Patent Application Publication 2015-0148341 (the publication of co-pending U.S. application Ser. No. 14/549,885, filed Nov. 21, 2014, which claims priority to U.S. Provisional Application Nos. 61/985,035, filed Apr. 28, 2014, and 61/908,333, filed Nov. 25, 2013), all of which patents, publications, and patent applications are hereby incorporated by reference herein in their entireties.

In some cases, the GRM having a non-steroidal backbone is a cyclohexyl pyrimidine (see, e.g., U.S. Pat. No. 8,685,973, hereby incorporated by reference in its entirety). In embodiments, wherein the cyclohexyl pyrimidine has the following formula:

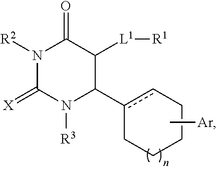

wherein the dashed line is absent or a bond; X is selected from the group consisting of O and S; R¹ is selected from the group consisting of cycloalkyl, heterocycloalkyl, aryl and heteroaryl, optionally substituted with from 1 to 3 R^{1a} groups; each R^{1a} is independently selected from the group consisting of H, C₁₋₆ alkyl, C₂₋₆ alkenyl, C₂₋₆ alkynyl, C₁₋₆ alkoxy, C₁₋₆ alkyl OR^{1b}, halogen, C₁₋₆ haloalkyl, C₁₋₆ haloaloxy, OR^{1b}, NR^{1b}R^{1c} C(O)R^{1b}, C(O)OR^{1b}, OC(O)R^{1b}, C(O)NR^{1b}R^{1c}, NR^{1b}C(O)R^{1c}, SO₂R^{1b} SO₂NR^{1b}R^{1c}, cycloalkyl, heterocycloalkyl, aryl and heteroaryl; R^{1b} and R^{1c} are each independently selected from the group consisting of H and C₁₋₆ alkyl; R² is selected from the group consisting of H, C₁₋₆ alkyl, C₁₋₆ alkyl-OR^{1b}, C₁₋₆ alkyl NR^{1b}R^{1c} and C₁₋₆ alkylene heterocycloalkyl; R³ is selected from the group consisting of H and C₁₋₆ alkyl; Ar is aryl, optionally substituted with 1-4 R⁴ groups; each R⁴ is independently selected from the group consisting of H, C₁₋₆ alkyl, C₁₋₆ alkoxy, halogen, C₁₋₆ haloalkyl and C₁₋₆ haloalkoxy; L¹ is a bond or C₁₋₆ alkylene; and subscript n is an integer from 0 to 3, or salts and isomers thereof.

In some cases, the GRM having a non-steroidal backbone is a fused ring azadecalin (see, e.g., U.S. Pat. No. 7,928,237, hereby incorporated by reference in its entirety). In embodiments, the fused ring azadecalin is a compound having the following formula:

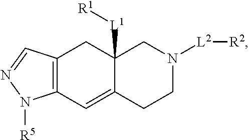

wherein L¹ and L² are members independently selected from a bond and unsubstituted alkylene; R¹ is a member selected from unsubstituted alkyl, unsubstituted heteroalkyl, unsubstituted heterocycloalkyl, —OR^{1A}, NR^{1C}R^{1D}, —C(O)NR^{1C}R^{1D}, and —C(O)OR^{1A}, wherein R^{1A} is a member selected from hydrogen, unsubstituted alkyl and unsubstituted heteroalkyl, R^{1C} and R^{1D} are members independently selected from unsubstituted alkyl and unsubstituted heteroalkyl, wherein R^{1C} and R^{1D} are optionally joined to form an unsubstituted ring with the nitrogen to which they are attached, wherein said ring optionally comprises an additional ring nitrogen; R² has the formula:

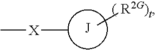

wherein R^{2G} is a member selected from hydrogen, halogen, unsubstituted alkyl, unsubstituted heteroalkyl, unsubstituted cycloalkyl, unsubstituted heterocycloalkyl, —CN, and —CF₃; J is phenyl; t is an integer from 0 to 5; X is —S(O₂)—; and R⁵ is phenyl optionally substituted with 1-5 R^{5A} groups, wherein R^{5A} is a member selected from hydrogen, halogen, —OR^{5A1}, S(O₂)NR^{5A2}R^{5A3}, —CN, and unsubstituted alkyl, wherein R^{5A1} is a member selected from hydrogen and unsubstituted alkyl, and R^{5A2} and R^{5A3} are members independently selected from hydrogen and unsubstituted alkyl, or salts and isomers thereof.

In some cases, the GRM having a non-steroidal backbone is a heteroaryl ketone fused azadecalin (see, e.g., U.S. Pat. No. 8,859,774, hereby incorporated by reference in its entirety). In embodiments, the heteroaryl ketone fused azadecalin has the formula:

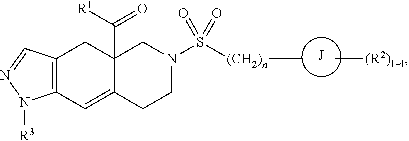

wherein R¹ is a heteroaryl ring having from 5 to 6 ring members and from 1 to 4 heteroatoms each independently selected from the group consisting of N, O and S, optionally substituted with 1-4 groups each independently selected from R^{1a}; each R^{1a} is independently selected from the group consisting of hydrogen, C₁₋₆ alkyl, halogen, C₁₋₆ haloalkyl, C₁₋₆ alkoxy, C₁₋₆ haloalkoxy, CN, N-oxide, C₃₋₈ cycloalkyl, and C₃₋₈ heterocycloalkyl; ring J is selected from the group consisting of a cycloalkyl ring, a heterocycloalkyl ring, an aryl ring and a heteroaryl ring, wherein the heterocycloalkyl and heteroaryl rings have from 5 to 6 ring members and from 1 to 4 heteroatoms each independently selected from the group consisting of N, O and S; each R² is independently selected from the group consisting of hydrogen, C₁₋₆ alkyl, halogen, C₁₆ haloalkyl, C₁₆ alkoxy, C₁₋₆ haloalkoxy, C₁₋₆ alkyl-C₁₋₆ alkoxy, CN, OH, NR^{2a}R^{2b}, C(O)R^{2a}, C(O)OR^{2a}, C(O)NR^{2a}R^{2b}, SR^{2a}, S(O)R^{2a}, S(O)₂R^{2a}, C₃₋₈ cycloalkyl, and C₃₋₈ heterocycloalkyl, wherein the heterocycloalkyl groups are optionally substituted with 1-4 R^{2c} groups; alternatively, two R² groups linked to the same carbon are combined to form an oxo group (═O); alternatively, two R² groups are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2d} groups; R^{2a} and R^{2b} are each independently selected from the group consisting of hydrogen and C₁₋₆ alkyl; each R^{2c} is independently selected from the group consisting of hydrogen, halogen, hydroxy, C₁₋₆ alkoxy, C₁₋₆ haloalkoxy, CN, and NR^{2a}R^{2b}; each R^{2d} is independently selected from the group consisting of hydrogen and C₁₋₆ alkyl, or two R^{2d} groups attached to the same ring atom are combined to form (═O); R³ is selected from the group consisting of phenyl and pyridyl, each optionally substituted with 1-4 R^{3a} groups; each R^{3a} is independently selected from the group consisting of hydrogen, halogen, and C₁₋₆ haloalkyl; and subscript n is an integer from 0 to 3; or salts and isomers thereof.

Exemplary GRMs having an octohydro fused azadecalin backbone include those described in U.S. Patent Application Publication 2015-0148341. Thus, in some cases, the GRM is an octahydro fused azadecalin having the formula:

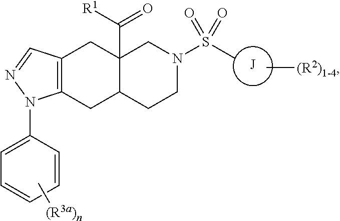

wherein R¹ is a heteroaryl ring having from 5 to 6 ring members and from 1 to 4 heteroatoms each independently selected from the group consisting of N, O and S, optionally substituted with 1-4 groups each independently selected from R^{1a}; each R^{1a} is independently selected from the group consisting of hydrogen, C₁₋₆ alkyl, halogen, C₁₋₆ haloalkyl, C₁₋₆ alkoxy, C₁₋₆ haloalkoxy, N-oxide, and C₃₋₈ cycloalkyl; ring J is selected from the group consisting of an aryl ring and a heteroaryl ring having from 5 to 6 ring members and from 1 to 4 heteroatoms each independently selected from the group consisting of N, O and S; each R² is independently selected from the group consisting of hydrogen, C₁₋₆ alkyl, halogen, C₁₋₆ haloalkyl, C₁₋₆ alkoxy, C₁₋₆ haloalkoxy, C₁₋₆ alkyl-C₁₋₆ alkoxy, CN, OH, NR^{2a}R^{2b}, C(O)R^{2a}, C(O)OR^{2a}, C(O)NR^{2a}R^{2b}, SR^{2a}, S(O)R^{2a}, S(O)₂R^{2a}, C₃₋₈ cycloalkyl, and C₃₋₈ heterocycloalkyl having from 1 to 3 heteroatoms each independently selected from the group consisting of N, O and S; alternatively, two R² groups on adjacent ring atoms are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2c} groups; R^{2a}, R^{2b} and R^{2c} are each independently selected from the group consisting of hydrogen and C₁₋₆ alkyl; each R^{3a} is independently halogen; and subscript n is an integer from 0 to 3, or salts and isomers thereof.

In particular embodiments, the GRM is the octahydro fused azadecalin compound (termed CORT125281) disclosed in Example 2C of U.S. Patent Publication 2015-0148341, having the chemical name: ((4aR,8aS)-1-(4-fluorophenyl)-6-((2-methyl-2H-1,2,3-triazol-4-yl)sulfonyl)-4,4a,5,6,7,8,8a,9-octahydro-1H-pyrazolo[3,4-g]isoquinolin-4a-yl)(4-(trifluoromethyl)pyridin-2-yl)methanone, which has the chemical formula:

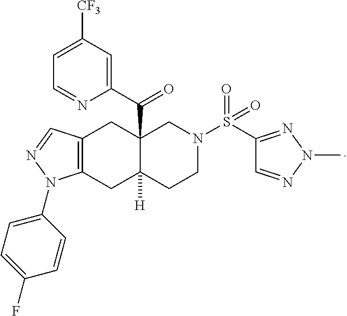 [SMILES: Cn1ncc(S(=O)(=O)N2CC[C@H]3Cc4c(cnn4-c4ccc(F)cc4)C[C@]3(C(=O)c3cc(C(F)(F)F)ccn3)C2)n1]

Further examples of non-steroidal GRMs include, for example N-(2-[4,4′,441-trichlorotrityl]oxyethyl)morpholine; 1-(2[4,4′,4″-trichlorotrityl]oxyethyl)-4-(2-hydroxyethyl)piperazine dimaleate; N—([4,4′,4″ ]-trichlorotrityl)imidazole; 9-(3-mercapto-1,2,4-triazolyl)-9-phenyl-2,7-difluorofluorenone; 1-(2-chlorotrityl)-3,5-dimethylpyrazole; 4-(morpholinomethyl)-A-(2-pyridyl)benzhydrol; 5-(5-methoxy-2-(N-methylcarbamoyl)-phenyl)dibenzosuberol; N-(2-chlorotrityl)-L-prolinol acetate; 1-(2-chlorotrityl)-1,2,4-triazole; 1, S-bis(4,4′,4″-trichlorotrityl)-1,2,4-triazole-3-thiol; 4α(S)-Benzyl-2(R)-chloroethynyl-1,2,3,4,4α,9,10,10α(R)-octahydro-phenanthrene-2,7-diol (“CP 394531”), 4α(S)-Benzyl-2(R)-prop-1-ynyl-1,2,3,4,4α,9,10,10α(R)-octahydro-phenanthrene-2,7-diol (“CP-409069”), trans-(1R,2R)-3,4-dichloro-N-methyl-N-[2-1 pyrrolidinyl)cyclohexyl] benzeneacetamide, bremazocine, and ethylketocyclazocine. Further non-steroidal GRMs suitable for use in GRM-containing pharmaceutical compositions as disclosed herein include, without limitation, e.g., the compound disclosed in Example 2A of U.S. Patent Application Publication 2015-0148341; the compound disclosed in Example 2B of U.S. Patent Application Publication 2015-0148341; the compound disclosed in Example 2D of U.S. Patent Application Publication 2015-0148341; the compound disclosed in Example 2E of U.S. Patent Application Publication 2015-0148341; the compound disclosed in Example 2N of U.S. Patent Application Publication 2015-0148341; the compound disclosed in Example 2P of U.S. Patent Application Publication 2015-0148341; the compound disclosed in Example 2R of U.S. Patent Application Publication 2015-0148341; the compound disclosed in Example 2AD of U.S. Patent Application Publication 2015-0148341; the compound disclosed in Example 2AJ of U.S. Patent Application Publication 2015-0148341; and other non-steroidal GRM compounds.

### Formulations

Formulations disclosed herein include formulations containing a GRM and other compounds which may aid in solubilizing the GRM, in stabilizing the GRM, in containing the GRM, or may be useful for other reasons in a pharmaceutical composition for oral administration.

Formulations suitable for use in the manufacture of pharmaceutical compositions for administration, including oral administration, to a patient and containing a GRM may include the GRM in proportions of between about 1% and about 50% by weight; in embodiments, such formulations and compositions containing a GRM may include the GRM in proportions of between about 3% and about 30% by weight; in embodiments, such formulations and compositions and containing a GRM may include the GRM in proportions of between about 5% and about 20% by weight; in embodiments, such formulations and compositions and containing a GRM may include the GRM in proportions of between about 6% and about 15% by weight; in further embodiments, such formulations and compositions and containing a GRM may include the GRM in proportions of between about 7% and about 14% by weight; and in further embodiments, such formulations and compositions and containing a GRM may include the GRM in proportions of between about 8% and about 12% by weight; and in embodiments, such formulations and compositions and containing a GRM may include the GRM in proportions of about 9%, of about 10%, and about 11% by weight.

Formulations suitable for use in the manufacture of pharmaceutical compositions for administration, including oral administration, to a patient and containing a GRM may include a solvent, solubilizer, or solubility enhancer, or a plurality of solvents, solubilizers, or solubility enhancers in proportions of between about 2% and about 90% by weight; in embodiments, such formulations and compositions containing a GRM may include a solvent, solubilizer, or solubility enhancer, or a plurality thereof, in proportions of between about 3% and about 80% by weight; or, in embodiments, in proportions of between about 5% and about 70% by weight; or in embodiments, in proportions of between about 8% and about 60% by weight; or, in further embodiments, in proportions of between about 10% and about 50% by weight. In embodiments, such formulations and compositions containing a GRM may not include a solvent.

Formulations suitable for use in the manufacture of pharmaceutical compositions for administration, including oral administration, to a patient and containing a GRM may include a surfactant, including a non-ionic surfactant, in proportions of between about 10% and about 90% by weight; in embodiments, such formulations and compositions containing a GRM may include surfactant, including a non-ionic surfactant, in proportions of between about 15% and about 80% by weight; in embodiments, such formulations and compositions containing a GRM may include surfactant, including a non-ionic surfactant, in proportions of between about 20% and about 75% by weight; and in embodiments, such formulations and compositions containing a GRM may include surfactant, including a non-ionic surfactant, in proportions of between about 25% and about 70% by weight. In embodiments, such formulations and compositions containing a GRM may not include a non-ionic surfactant. In embodiments, such formulations and compositions containing a GRM may not include a surfactant.

In embodiments, formulations suitable for use in the manufacture of pharmaceutical compositions for administration, including oral administration, to a patient and containing a GRM may include a polysorbate (such as, e.g., Tween® 20 or Tween® 80) in proportions of between about 5% and about 50% by weight; and in embodiments, such formulations and compositions containing a GRM may include a polysorbate in proportions of between about 10% and about 40% by weight. In embodiments, such formulations and compositions containing a GRM may not include a polysorbate.

In embodiments, formulations suitable for use in the manufacture of pharmaceutical compositions for administration, including oral administration, to a patient and containing a GRM may include a polyethylene glycol (PEG) in proportions of between about 5% and about 50% by weight; in embodiments, such formulations and compositions containing a GRM may include a PEG in proportions of between about 10% and about 40% by weight; and in embodiments, such formulations and compositions containing a GRM may include a PEG in proportions of between about 15% and about 30% by weight.

### Exemplary Formulations

Many GRMs, including many non-steroidal GRMs, are difficult to formulate in pharmaceutically acceptable formulations. Problems include poor solubility in water; poor solubility in solvents that may be used in pharmaceutical formulations; poor stability; low bioavailability; incompatibility with pharmaceutically acceptable capsules and coatings; and other difficulties.

For example, a formulation suitable for oral administration and containing a GRM may contain 11% GRM, 8.9% Transcutol HP, 35.6% Gelucire, 31.2% Vitamin E (VitE), and 13.3% PEG400 (where % indicates % w/w).

For example, a formulation suitable for oral administration and containing a GRM may contain 11% GRM, 20% Transcutol HP, 30.65% Gelucire, 26.85% Vitamin E TPGS, and 11.5% PEG400 (where % indicates % w/w).

A further formulation suitable for oral administration and containing a GRM may contain 11% GRM, 10% Transcutol HP, 49% Gelucire 44/14, and 30% Kolliphor (where % indicates % w/w).

A further formulation suitable for oral administration and containing a GRM may contain 11% GRM, 29.5% Kolliphor HS15, 39.5% Vitamin E, and 20% PEG400 (where % indicates % w/w).

A further formulation suitable for oral administration and containing a GRM may contain 11% GRM, 35.6% Capryol 90, 26.7% Tween 20, and 26.7% Tween 80 (where % indicates % w/w).

A further formulation suitable for oral administration and containing a GRM may contain 11% GRM, 17.8% Medium Chain Triglycerides, 44.5% Tween 20, and 26.7% Kolliphor RH40 (where % indicates % w/w).

A further formulation suitable for oral administration and containing a GRM may contain 11% GRM, 20% Transcutol HP, and 69% Vitamin E TPGS (where % indicates % w/w).

A further formulation suitable for oral administration and containing a GRM may contain 10% GRM, 9% Transcutol HP, 27% Kolliphor HS 15, and 54% Labrasol (where % indicates % w/w).

A further formulation suitable for oral administration and containing a GRM may contain 11% GRM, 8.9% Transcutol HP, 10% Triacetin, 20% Kolliphor RH40, and 50.1% Gelucire 44/14 (where % indicates % w/w).

A further formulation suitable for oral administration and containing a GRM may contain 11% GRM, 8.9% Transcutol HP, 21.1% Triacetin, and 59% Vitamin E TPGS (where % indicates % w/w).

A further formulation suitable for oral administration and containing a GRM may contain 11% GRM, 8.9% Transcutol HP, 11.1% Triacetin, and 69% Vitamin E TPGS (where % indicates % w/w).

A further formulation suitable for oral administration and containing a GRM may contain 11% GRM, 8.9% Transcutol HP, 17.0% Triacetin, and 63.1% Vitamin E TPGS (where % indicates % w/w).

A further formulation suitable for oral administration and containing a GRM may contain 8.8% GRM, 9.12% Transcutol HP, 36.48% Gelucire 44/14, 13.63% PEG400, and 31.97% Vitamin E TPGS (where % indicates % w/w).

In embodiments of the formulations disclosed herein, including in embodiments of the exemplary formulations disclosed above, the GRM is a non-steroidal GRM. In embodiments of the formulations disclosed herein, including in embodiments of the exemplary formulations disclosed above, the GRM is a non-steroidal glucocorticoid receptor modulator disclosed in U.S. Pat. No. 7,576,076; U.S. Pat. No. 7,678,813; U.S. Pat. No. 7,928,237; U.S. Pat. No. 8,461,172; U.S. Pat. No. 8,598,154; U.S. Pat. No. 8,685,973; U.S. Pat. No. 8,859,774; U.S. Pat. No. 8,889,867; U.S. Pat. No. 9,321,736; or U.S. Patent Publication 2015-0148341.

In particular embodiments of the formulations disclosed herein, including in particular embodiments of the exemplary formulations disclosed above, the GRM is a non-steroidal GRM disclosed in U.S. Patent Publication 2015-0148341. In yet more particular embodiments of the formulations disclosed herein, including in yet more particular embodiments of the exemplary formulations disclosed above, the GRM is CORT125281.

### Pharmaceutical Compositions

Applicant discloses herein compositions comprising a glucocorticoid receptor modulator (GRM) may be useful in treating patients suffering from a condition amenable to treatment with a GRM, and may be used in the treatment of a patient in need of such treatment. Conditions amenable to treatment with a GRM may include without limitation, for example, Cushing's syndrome, Cushing's Disease, pancreatic cancer, prostate cancer, breast cancer, ovarian cancer, other hormone-sensitive cancer, other cancer, liver disease (including a fatty liver disease, fibrosis, cirrhosis, and other liver diseases), depression, dementia, stress disorders (including post-traumatic stress disorders, anxiety, and other stress disorders), substance abuse disorders, and other diseases, disorders, and conditions.

The compositions as disclosed herein can be prepared in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions disclosed herein can be administered by inhalation, for example, intranasally. Additionally, the compositions of the present invention can be administered transdermally. The compositions disclosed herein can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995).

Accordingly, in embodiments disclosed herein, the compositions include pharmaceutical compositions including a pharmaceutically acceptable carrier or excipient, and a glucocorticoid receptor modulator (GRM).

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Mack Publishing Co, Easton Pa. (“Remington's”).

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from about 2% to about 70% of the GRM.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain the GRM mixed with a filler or binders such as lactose or starches, lubricants such as talc or magnesium stearate, and, optionally, stabilizers. In soft capsules, the GRM may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the GRM are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the GRM in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the GRM in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present invention can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In another embodiment, the compositions of the present invention can be formulated for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

### Administration

Methods of treating diseases including Cushing's syndrome and hormone-sensitive cancers by administration of a GRM in such pharmaceutical compositions are also provided. In embodiments, a patient suffering from a disease or condition amenable to treatment by the GRM is administered a pharmaceutical composition disclosed herein; in embodiments, the administration comprises oral administration.

The compositions disclosed herein can be delivered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

The pharmaceutical preparation is preferably in unit dosage form. In such form the preparation is subdivided into unit doses containing appropriate quantities of the GRM. In embodiments, the GRM is a non-steroidal GRM. In embodiments, the GRM is CORT125281.

In embodiments, a unit dosage form contains between about 0.1 milligram (mg) of the GRM and about 750 mg of the GRM; in further embodiments, a unit dosage form contains between about 1 mg of the GRM and about 600 mg of the GRM; in further embodiments, a unit dosage form contains between about 5 mg of the GRM and about 500 mg of the GRM; in further embodiments, a unit dosage form contains between about 10 mg of the GRM and about 400 mg of the GRM; in further embodiments, a unit dosage form contains between about 15 mg of the GRM and about 350 mg of the GRM; in further embodiments, a unit dosage form contains between about 20 mg of the GRM and about 300 mg of the GRM; in further embodiments, a unit dosage form contains between about 25 mg of the GRM and about 250 mg of the GRM. In embodiments, the GRM is a non-steroidal GRA.

In particular embodiments, the GRM is CORT125281. In embodiments in which the GRM is CORT125281, the unit dosage form may contain between about 5 milligrams (mg) and about 150 mg CORT125281. In further embodiments in which the GRM is CORT125281, the unit dosage form may contain between about 10 mg and about 100 mg CORT125281. In yet further embodiments in which the GRM is CORT125281, the unit dosage form may contain between about 15 mg and about 75 mg CORT125281. In still further embodiments in which the GRM is CORT125281, the unit dosage form may contain between about 20 mg and about 60 mg CORT125281. In yet further embodiments in which the GRM is CORT125281, the unit dosage form may contain between about 30 mg and about 50 mg CORT125281. In particular embodiments in which the GRM is CORT125281, the unit dosage form may contain about 40 mg CORT125281.

The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The GRM can be administered once a day, or two, three, or more times per day so as to provide the preferred dosage level per day. In embodiments, the GRM is a non-steroidal GRM. In particular embodiments, the non-steroidal GRM is CORT125281.

The composition can also contain other compatible therapeutic agents. The compounds described herein can be used in combination with one another, or with adjunctive agents that may not be effective alone, but may contribute to the efficacy of the active agent.

### Kits

Applicant further provides kits including compositions as disclosed herein. Kits may also include instructions for the use of the compositions.

In embodiments, a kit includes: a pharmaceutical composition containing a GRM. In embodiments, the GRM is a non-steroidal GRM. In particular embodiments, the non-steroidal GRM is CORT125281.

In embodiments, a kit includes: a pharmaceutical composition the GRM; and instructions for the use (e.g., administration) of the GRM. In embodiments, the GRM is a non-steroidal GRM, and the instructions include instructions for the administration of the non-steroidal GRM. In embodiments, the GRM is CORT125281, and the instructions include instructions for the administration of CORT125281. In embodiments, the instructions include instructions regarding one or more of the number of pharmaceutical compositions to be taken each day, the timing of such administration, whether or not the pharmaceuticals are to be taken with food or in a fasted state, contraindications, possible side effects, activities to be avoided during treatment with the pharmaceutical compositions (if any), and foods to be avoided during treatment with the pharmaceutical compositions (if any).

In embodiments, a kit includes: a pharmaceutical composition containing a GRM; and instructions for the use (e.g., administration) of the pharmaceutical composition. In embodiments, the GRM is a non-steroidal GRM. In particular embodiments, the non-steroidal GRM is CORT125281. In embodiments of the kits disclosed herein, the pharmaceutical composition includes a non-steroidal GRM, and the instructions include instructions for the administration of the pharmaceutical containing the non-steroidal GRM. In embodiments of the kits disclosed herein, the pharmaceutical composition includes CORT125281, and the instructions include instructions for the administration of the pharmaceutical containing CORT125281. In embodiments, the instructions include instructions regarding one or more of the number of pharmaceutical compositions to be taken each day, the timing of such administration, whether or not the pharmaceutical composition is to be taken with food or in a fasted state, contraindications, possible side effects, activities to be avoided during treatment with the pharmaceutical composition (if any), and foods to be avoided during treatment with the pharmaceutical composition (if any).

### EXAMPLES

The following examples are presented by way of illustration of embodiments of the methods disclosed herein, and serve to illustrate, but not to limit, the present disclosure of formulations containing GRMs.

Pharmaceutical formulations may be encapsulated in capsules; for example, the capsules may be hard gelatin capsules (e.g., Coninsnap®, available from Capsugel, Morristown, N.J., USA); hydroxypropyl methylcellulose capsules (e.g., Vcaps Plus®, available from Capsugel, Morristown, N.J., USA); or other capsule type. Capsules typically are sized to as to accommodate up to one or a few milligrams of the active pharmaceutical ingredient (i.e., the GRM, which may be a non-steroidal GRM, such as, e.g., CORT125281) and accompanying excipients. Capsules suitable for use include size 4 capsules; size 3 capsules; size 2 capsules; size 1 capsules; size 0 capsules; size 00 capsules; and other size capsules. In embodiments, a unit dosage of CORT125281 is contained in a capsule. In embodiments, a unit dosage of CORT125281 is contained in a size 2 capsule. In embodiments, a unit dosage of CORT125281 is contained in a size 1 capsule. In embodiments, a unit dosage of CORT125281 is contained in a size 0 capsule. In embodiments, a unit dosage of CORT125281 is contained in a size 00 capsule.

In the following examples, excipients included: Transcutol HP; Gelucire 44/14; include α-tocopherol polyethylene glycol succinate (Vitamin E TPGS); polyethylene glycol 400 (PEG400) and polyethylene glycol (PEG) of other weights; Kolliphor RH40; Kolliphor HS15; Tween 20; Tween 80; Capryol 90; and Medium Chain Triglycerides (MCT). Banding solution components include sterile water for irrigation; Gelatin 220 Bloom; Pharmacoat 603; and ethanol (banding solution is used to seal the capsule after filling).

In the following, the capsule shell types used included Coninsnap® (available from Capsugel, Morristown, N.J., USA) and Vcaps Plus® (available from Capsugel, Morristown, N.J., USA). Capsules may be sealed with a band, such as a gelatin band, to provide the capsule with a tamper-resistant seal.

As noted above, many GRMs, including many non-steroidal GRMS, are poorly soluble in pharmaceutically acceptable compositions and solvents; in addition, pharmaceutical formulations of many such GRMs, including many such non-steroidal GRMS, often provide only poor bioavailability. Many GRMs, including many non-steroidal GRMS, are difficult to formulate so as to provide acceptable stability. Due to the requirements for solubilization, and the solvents needed for such solubilization, many GRMs, including many non-steroidal GRMS, are difficult to formulate so as to provide acceptable biocompatibility of the pharmaceutical formulation (e.g., to provide formulations which do not include, or only include minimal amounts of, excipients which may have uncomfortable, adverse, or toxic effects on the subject to which they are administered).

The following examples discuss several exemplary formulations prepared as discussed herein. All of the formulations discussed herein are believed to provide useful amounts of solubilization of the active ingredient, and to provide sufficient formulation stability, including sufficient stability of the active ingredient, so as to be suitable for use in the administration of pharmaceutically active compounds to human subjects. In particular, all of the formulations discussed herein are believed to provide useful amounts of solubilization of a non-steroidal GRM, and to provide sufficient formulation stability, including sufficient stability of the non-steroidal GRM, so as to be suitable for use in the administration of non-steroidal GRM compounds to human subjects. In addition, the formulations disclosed herein are believed to provide improved bioavailability of the non-steroidal GRM active ingredient as compared to prior or alternative formulations. The formulations including non-steroidal GRM active ingredients disclosed herein are further believed to provide improved biocompatibility as compared to prior or alternative formulations. The formulations disclosed herein, being suitable for use in the administration of non-steroidal GRM compounds to human subjects, are believed to be suitable for use in the treatment of human subjects suffering from disorders amenable to treatment by non-steroidal GRM compounds. The formulations disclosed herein are believed to provide improved pharmaceutical formulations which solve the problems of poor solubility, or poor bioavailability, or poor biocompatibility, or poor stability, previously encountered with prior or alternative formulations of non-steroidal GRM compounds for administration to human subjects.

### Example 1

Formulation preparation—formulations containing an active ingredient (i.e., the non-steroidal GRM CORT125281) were prepared with the proportions and amounts shown in Table 1; typical batch size was 50 gram (g) or 150 g, unless the size was altered depending on the material requirements for testing or for solubilization. Each batch provided a sufficient amount of formulation for use in filling multiple capsules.

If necessary, the GRM (the active pharmaceutical ingredient in the formulations) is not solubilized at 11%, additional excipient may be added to attain full solubilization.

Equipment used in the procedures discussed herein (e.g., in the Examples) included balances, laboratory grade glassware, a fan oven, laboratory mixers, stirrers (e.g., multiplate magnetic stirrers), vacuum desiccators, vacuum chambers, temperature probes, band-applying machines (e.g., Quali-Seal™ capsule sealing machine, ELANCO Qualicaps, Indianapolis, Ind., US), viscometers (e.g., Brookfield DVIII Viscometer, Brookfield Engineering Laboratories, Middleboro, Mass., USA), calorimeters (e.g., Perkin Elmer DSC6000 Differential Scanning Calorimeter, Perkin Elmer, San Jose Calif., USA), stability cabinets (e.g., cabinets that maintain temperature and humidity at, e.g., 25° C. and 60% relative humidity (R.H.), or, e.g., at 40° C. and 75% R.H.), and other laboratory equipment.

Formulations have been, and may be prepared according to the following procedures:
i. Heat thermosoftening excipients until molten at the required melting temperature: Vitamin E TPG5: melting range 37-41° C.; Kolliphor RH40: 60° C., Kolliphor HS 15: 60-65° C.; and Gelucire 44/14: 70-80° C. All excipients were homogenized before use.
ii. Weigh the appropriate quantity of excipients into a labelled vessel.
iii. Add CORT125281 (the active ingredient) to the labelled vessel and mix manually, ensuring the powder is thoroughly wetted,
iv. High shear the sample using the laboratory mixer with small head attachment until a visibly clear solution has been achieved. Light microscopy may be used to confirm that a solution has been obtained. The high shear mixing time should not exceed individual periods of 5 minutes. Record the mixing time duration and the temperature of the mix prior to and after mixing. If a solution is unable to be achieved, further solubilization techniques may be used.
v. Proceed to degas the formulation in a vacuum desiccator. Degassing is complete when there are no bubbles remaining and the bulk mix has returned to its original volume.
vi. Assess the physical stability of the formulation.

Viscosity Testing: The viscosity of formulations was assessed using a Brookfield DV-111 Ultra Programmable Rheometer. A suitable assessment method was used and details of these methods were recorded in the laboratory notebook. Any formulations which are liquid at room temperature were measured at 25° C. All other formulations were measured at 55° C.

The formulations were tested by Dispersion Testing. In such testing, formulations (including both active formulations (containing a GRM) and corresponding placebo formulations (lacking a GRM, but otherwise the same as the formulations containing a GRM) may be assessed. Both active and corresponding placebo formulations may be assessed using dispersion testing. The formulations discussed herein were assessed as discussed.

Placebo formulations were also prepared. The preparation of placebo formulations was as described for the preparation of active formulations (see steps i-vi above), with the exception that step iii, the addition of the active ingredient (CORT125281) was omitted. The batch size was typically 25 g, but may be altered if desired depending on the materials required. The following procedure was used for the preparation of placebo formulations:
i. Weigh 1 gram of the formulation into a beaker and add 10 milliliter (ml) of water. Visually assess the dispersion characteristics on stirring.
ii. Increase the water gradually to a total of 250 ml using increments of 20 ml, 30 ml, 40 ml, 50 ml, 75 ml, 100 ml, 150 ml, 200 ml and 250 ml. After each addition of water visually assess the dispersion characteristics on stirring.
iii. When the volume has reached 250 ml gently mix the dispersion for 6 hours at 37° C., visually assessing the dispersion characteristics, including any observed sedimentation of the dispersion.
iv. In parallel weigh 1 gram (g) of placebo control formulation into a beaker and repeat steps 1 to 3.
v. After agitating for 6 hours, stop mixing and assess the sedimentation characteristics of the dispersions.
vi. Reassess the sedimentation characteristics of the dispersions after approximately 24 hours of storage at ambient conditions on the bench.

Filling and Banding of Capsules

The target bulk mix weight was filled into size 1 hydroxypropyl methylcellulose (HPMC) and gelatin capsules to a target fill-weight of 364 mg (limits: 336.7-391.3 mg). In some instances, when necessary for solubilization, a larger capsule shell was used when drug loading was required to be reduced to permit solubilization in formulations 4 and 5. Capsules were filled and banded as detailed below:

Filling and Banding Procedure:
i. Prepare a 25% gelatin banding solution in sterile water.
ii. Prepare a 16% Pharmacoat banding solution, in 64% ethanol (96%) and 20% sterile water.
iii. Fill approximately 40 size 0 gelatin capsules with 364 mg of formulation 1 and band capsules with gelatin banding solution using the bench Qualiseal banding machine.
iv. Fill approximately 40 size 0 HPMC capsules with 364 mg of formulation 1 and band capsules with HPMC banding solution using the bench Qualiseal banding machine.
v. Repeat steps i-iv, filling with the remaining formulations.
vi. Allow the banded capsules to dry for a minimum of 8 hours on a stainless steel tray.
vii. Visually inspect all capsules. Remove any defective capsules and record any observations in the laboratory notebook.

Vacuum test the banded capsules. Remove any leaking capsules from the batch recording the reason for leaking where possible.

Capsule Shell Compatibility Assessment

The compatibility of the capsule shell and the several formulations were assessed as follows:
i. Pack N 20 capsules (HPMC or gelatin) per dose strength (placebo, active) into a labelled amber glass jar.
ii. Place in a stability cabinet at 40° C. and 75% R.H. for two weeks.
iii. Repeat steps i. and ii., placing capsules in a stability cabinet at 25° C. and 60% R H. for two weeks.
iv. Remove the capsules from storage after two weeks and assess the capsules for leaking, cracking and embrittlement, recording any observations in the laboratory notebook

Differential Scanning Calorimetry (DSC) Analysis

DSC analysis was performed on a Perkin Elmer DSC6000 Differential Scanning Calorimeter in order to provide an indication of the stability of the molecule in the amorphous state as well as its propensity to recrystallization. The DSC analysis Procedure was as follows:
a) Weigh 2 to 6 mg of CORT125281 into an aluminum sample pan (n=2), immediately sealing the pan.
b) Weigh 2 to 6 mg of CORT125281 into an aluminum sample pan (n=2), recording the weight of the empty pan, and leave at ambient conditions overnight prior to sealing.
c) Purge the furnace with N2 (flow rate 20 ml per min) and use an empty pan to apply a baseline correction.
d) Apply the following method to both samples:
1. Heat from 25° C.-200° C. at 10° C. per min.
2. Hold for 10 minutes at 200° C.
3. Cool from 200° C. to 10° C. at 5° C. per min.
4. Hold for 1 minute at 10° C.
5. Re-heat from 10° C. to 200° C. at 10° C. per min.

The formulations and measurements discussed above were used, in part, to: Prepare the formulation bulk mixes; Perform viscosity testing on formulation bulk mixes; Perform dispersion testing of formulation bulk mixes; Fill formulation bulk mixes into gelatin and HPMC capsule shells and assess the physical stability of the capsules following storage at ambient and accelerated conditions; and Perform DSC analysis on CORT125281 to assess glass transition (T,) and melting (Tm) temperatures.

An exemplary initial formulation containing CORT125281 was developed and tested according to the procedures discussed herein. The initial exemplary formulation containing CORT125281 suitable for oral administration contained (by weight) 11% CORT125281, 8.9% Transcutol HP, 35.6% Gelucire, 31.2% Vitamin E, and 13.3% PEG400 (where % indicates % w/w) as provided in the following Table 1C. This formulation served as a basis for further modifications of the formulations containing CORT125281.

TABLE 1C
Initial Exemplary Formulation with CORT125281
11.00%
CORT125281
8.9%
Transcutol HP
35.6%
Gelucire
31.2%
Vitamin E TPGS
13.3%
PEG400

### Example 2

Following the work discussed above, and guided, e.g., by the information obtained from the formulations discussed above, further formulations containing CORT125281 were developed and prepared as discussed below. These further formulations containing CORT125281 (also termed “active formulations”) were made according to the methods disclosed herein, and in the proportions provided in the following examples (and tabulated in TABLE 2A, with corresponding weights for formulations 1 to 7 presented in TABLE 2B); placebo formulations are presented in TABLE 3.

TABLE 2A
Further CORT125281 Formulations

| 1 | 2 | 3 | 4 | 5 | 6 | 7 | 8 | 9 | 10 |
|---|---|---|---|---|---|---|---|---|---|
| 11.00% | 11.00% | 11.00% | 11.00% | 11.00% | 11.00% | 10.00% | 11.00% | 11.00% | 11.00% |
| CORT | CORT | CORT | CORT | CORT | CORT | CORT | CORT | CORT | CORT |
| 125281 | 125281 | 125281 | 125281 | 125281 | 125281 | 125281 | 125281 | 125281 | 125281 |
| 20.0% | 10.0% | 20.00% | 35.60% | 17.80% | 20.00% | 9.00% | 8.90% | 8.90% | 8.90% |
| Transcutol | Transcutol | PEG400 | Capryol 90 | Medium Chain | Transcutol | Transcutol | Transcutol | Transcutol | Transcutol |
| HP | HP | | | Triglycerides | HP | | HP | HP | HP |
| 30.65% | 30.0% | 39.50% | 26.70% | 44.50% | 69.00% | 54.00% | 20.0% | 59.00% | 69.00% |
| Gelucire | Kolliphor | Vitamin E | Tween 20 | Tween 20 | Vitamin E | Labrasol | Kolliphor | Vitamin E | Vitamin E |
| | | TPGS | | | TPGS | | RH40 | TPGS | TPGS |
| 26.83% | 49.00% | 29.50% | 26.70% | 26.70% | — | 27.00% | 10.0% | 21.1% | 11.1% |
| Vitamin E | Gelucire | Kolliphor | Tween 80 | Kolliphor | | Kolliphor | Triacetin | Triacetin | Triacetin |
| TPGS | 44/14 | HS15 | | RH40 | | HS15 | | | |
| 11.50% | — | — | — | — | — | — | 50.1% | | |
| PEG400 | | | | | | | Gelucirc | | |
| | | | | | | | 44/14 | | |

TABLE 2B
Weights for CORT125281 Formulations 1-7

| | 1 | | 2 | | 3 | | 4 | | 5 | | 6 | | 7 | |
|---|---|---|---|---|---|---|---|---|---|---|---|---|---|---|
| | 16.50 | g | 16.50 | g | 16.50 | g | 16.50 | g | 16.50 | g | 16.50 | g | 15.0 | g |
| | CORT125281 | | CORT125281 | | CORT125281 | | CORT125281 | | CORT125281 | | CORT125281 | | CORT125281 | |
| | 30.00 | g | 15.0 | g | 30.00 | g | 53.40 | g | 26.70 | g | 103.50 | g | 13.50 | |
| | Transcutol | | Transcutol | | PEG400 | | Capryol 90 | | Medium Chain | | Vitamin E | | Transcutol | |
| | HP | | HP | | | | | | Triglycerides | | TPGS | | HP | |
| | 45.975 | g | 45.0 | g | 59.250 | g | 40.05 | g | 66.75 | g | 30.00 | g | 81.00 | g |
| | Gelucire | | Kolliphor | | Vitamin E | | Tween 20 | | Tween 20 | | Transcutol | | Labrasol | |
| | 44/14 | | RH40 | | TPGS | | | | | | HP | | | |
| | 40.275 | g | 73.50 | g | 44.250 | g | 40.05 | | 40.05 | g | — | | 40.500 | g |
| | Vitamin E | | Gelucire | | Kolliphor | | Tween 80 | | Kolliphor | | | | Kolliphor | |
| | TPGS | | 44/14 | | HS15 | | | | RH40 | | | | HS15 | |
| | 17.250 | g | — | | — | | — | | — | | — | | — | |
| | PEG400 | | | | | | | | | | | | | |
| TOTAL | 150 | g | 150 | g | 150 | g | 150 | g | 150 | g | 150 | g | 150 | g |

TABLE 3
Placebo Formulations

| | 1 | | 2 | | 3 | | 4 | | 5 | | 6 | | 7 | |
|---|---|---|---|---|---|---|---|---|---|---|---|---|---|---|
| | 5.625 | g | 2.800 | g | 5.625 | g | 10.000 | g | 5.000 | g | 19.300 | g | 2.500 | g |
| | Transcutol | | Transcutol | | PEG400 | | Capryol 90 | | Medium Chain | | Vitamin E | | Transcutol | |
| | HP | | HP | | | | | | Triglycerides | | TPG5 | | HP | |
| | 8.600 | g | 8.425 | g | 11.100 | g | 7.500 | g | 12.500 | g | 5.625 | g | 15.000 | g |
| | Gelucire | | Kolliphor | | Vitamin E | | Tween 20 | | Tween 20 | | Transcutol | | Labrasol | |
| | 44/14 | | RH40 | | TPGS | | | | | | HP | | | |
| | 7.550 | g | 13.775 | g | 8.275 | g | 7.500 | g | 7.500 | g | — | | 7.500 | g |
| | Vitamin E | | Gelucire | | Kolliphor | | Tween 80 | | Kolliphor | | | | Kolliphor | |
| | TPGS | | 44/14 | | H515 | | | | RH40 | | | | HS 15 | |
| | 3.225 | g | — | | — | | — | | — | | — | | — | |
| | PEG400 | | | | | | | | | | | | | |
| Total | 25.000 | g | 25.000 | g | 25.000 | g | 25.000 | g | 25.000 | g | 25.000 | g | 25.000 | g |

Results

### Solubility Assessment of CORT125281 in Various Excipients.

The solubility of the API was assessed in Labrasol, Kolliphor RH40, Kollisolv P124 and Kolliphor HS15 in order to assess scope for formulation alternatives. It was found that solubility at 40 mg/g was not achieved with Kolliphor RH40 and Kollisolv P124. Labrasol and Kolliphor HS15 mixes were visibly clear, but upon microscopic analysis, they were found to contain a small number of API crystals. It is probable that these would be able to be solubilized given more time/energy input.

Seven placebo formulations were prepared without any issues and produced solutions that were transparent and free from any visible particles. Seven active formulations were prepared and full dissolution was only achieved with active formulations 1, 6 and 7. The results are summarized in Table 4.

TABLE 4
Results of drug solubilization
(“API” indicates the active pharmaceutical ingredient, i.e., CORT125281)

| Formulation | Comments |
|---|---|
| Formulation 1 | After a total of 21 minutes high shear mixing with a maximum temperature of |
| | 60.5° C. achieved, 90 minutes of sonication and heating at 55° C. for 48 hours the |
| | API was visually soluble. Full solubilization was achieved. |
| Formulation 2 | After a total of 21 minutes high shear mixing (maximum temperature of 61.0° C. |
| | recorded), 90 minutes of sonication and heating at 55° C. for 48 hours the mix still |
| | had some API settled on the bottom of the vessel. Full solubilization not |
| | achieved. |
| Formulation 3 | After a total of 21 minutes high shear mixing (maximum temperature of 60.8° C. |
| | recorded), 90 minutes of sonication and heating at 55° C. for 48 hours the mix still |
| | had some API settled on the bottom of the vessel. |
| Formulation 4 | After a total of 11 minutes high shear mixing (maximum temperature of 33.1° C. |
| | recorded) and 30 minutes of sonication powder was still visible on the bottom of |
| | the vessel. The mix had extra excipient added to reduce the drug load to 8.3%. |
| | The bulk mix was then high sheared for a further 15 minutes and sonicated for |
| | 60 minutes after which API still remained insoluble. |
| Formulation 5 | After 6 minutes of high shear mixing (maximum temperature of 53.9° C. |
| | achieved), 30 minutes of sonication and overnight hold at 55° C., the majority of |
| | the API was not solubilized. Further excipients were added to give a drug load of |
| | 8.3% and the mix given a further 15 minutes high shear mixing (21 minutes |
| | total, maximum temperature of 61.1° C.) and 60 minutes of sonication, a |
| | significant quantity of powder was still not solubilized. |
| Formulation 6 | After 6 minutes of high shear mixing (maximum temperature of 51.8° C. |
| | achieved), 30 minutes of sonication and overnight hold at 55° C., some API had |
| | not dissolved. The mix was high sheared for a further 15 minutes (21 minutes |
| | total, maximum temperature of 60.7° C. achieved), and sonicated for 60 minutes |
| | (90 minutes total) after which the API appeared to be fully solubilized. |
| Formulation 7 | After 6 minutes of high shear mixing (maximum temperature of 50.7° C. |
| | achieved) and 30 minutes of sonication, some API remained insoluble. Bulk mix |
| | was held overnight at 55° C.; visual assessment the following day showed some |
| | API remained insoluble. Bulk mix was high shear mixed for a further 15 minutes |
| | (21 minutes total, maximum temperature of 60.0° C. achieved) and sonicated for |
| | 60 minutes (90 minutes total) prior to holding at 55° C. for 60 minutes. Upon |
| | visual assessment, the mix appeared fully solubilized. |

Based on the data obtained here, formulations 1, 6 and 7 were taken forward for further evaluation.

### Viscosity Testing Results

Viscosity testing results for active formulations 1, 6, 7 and the initial exemplary formulation were obtained. All tests were carried out at 55° C., i.e. in the molten state.

The initial exemplary formulation exhibits Newtonian behavior with viscosity being independent of shear rate across a shear rate of 75 to

$$600 \frac{1}{sec} .$$

The mean viscosity was calculated as 80.71 cP (range 79.77-83.38 cP; standard deviation of 1.23). If values at

$$15.00 \frac{1}{sec} .$$

are discounted the mean viscosity becomes 80.61 cP (range 79.77-81.10 cP; standard deviation of 0.38). The viscosity range is suitable for filling of hard capsules.

The viscosity profile for active formulation 1 exhibits Newtonian behavior with viscosity being independent of the shear rate across a shear rate of 75 to

$$450 \frac{1}{sec} .$$

The mean viscosity was calculated as 48.92 cP with a range of 48.67 P to 49.21 cP (standard deviation of 0.20). The viscosity range is suitable for filling of hard capsules.

Active formulation 6 exhibits Newtonian behavior, with viscosity being independent of the shear rate across a shear rate of 75 to

$$225 \frac{1}{sec} .$$

The mean viscosity was calculated as 96.71 cP (range 94.83-97.77 cP; standard deviation 0.78). The initial shear rate of

$$15.0 \frac{1}{sec}$$

could be discounted as not being representative as it is possible the shear rate was approaching the lower limit of the range of the method or the sample had not fully warmed at this point. The viscosity range is suitable for filling of hard capsules.

Active formulation 7 exhibits Newtonian behavior, with viscosity being independent of shear rate across a shear rate of 75 to

$$600 \frac{1}{sec} .$$

The mean viscosity was calculated as 32.99 cP (range 32.37-33.24 cP; standard deviation of 0.29). The viscosity range is suitable for filling of hard capsules.

### Active and Placebo Formulation Dispersion Testing

Active formulations 1, 6 and 7 were tested along with the corresponding placebos for their dispersion characteristics, and the results are summarized in Table 5. The dispersion test suggests that generally all active formulations performed similarly, with active formulations 6 and 7 being somewhat less prone to drug settling than active formulation 1.

TABLE 5

| | | | After 24 Hours |
|---|---|---|---|
| Formulation | During Dispersion | After 6 Hours Mixing | Settling |
| Active 1 | Fine particulate matter | Suspension was opaque, | Suspension clear |
| | evident throughout all | some frothing with some | and colorless, |
| | volume levels. Suspension | fine particulate matter still | with all |
| | was opaque with some | evident. Material was fully | particulate |
| | frothing. Crystals were | dispersed throughout the | material settled |
| | observed on the side of the | suspension | out. |
| | beaker at 150 ml dilution. | | |
| Placebo 1 | Fully dispersed, with no | Fully dispersed, with no | No visible |
| | particulate matter visible, | particulate matter visible, | precipitate or |
| | | | phase separation |
| | | | evident. |
| Active 6 | Suspension was opaque with | Suspension was opaque | Suspension clear |
| | some frothing. Material was | with some frothing. | and colorless, |
| | fully dispersed throughout, | Material was fully dispersed | with all material |
| | with no sign of crystals, | throughout the suspension. | settled out. |
| | some air bubbles on surface | | |
| | at the 150 ml dilution point. | | |
| Placebo 6 | Fully dispersed, with no | Fully dispersed, with no | No visible |
| | particulate matter visible, | particulate matter visible, | precipitate or |
| | | | phase separation |
| | | | evident. |
| Active 7 | Suspension was opaque with | Suspension was opaque | Suspension clear |
| | some frothing. There was no | with some frothing. | and colorless, |
| | sign of crystals at the 150 ml | Material was fully dispersed | with all material |
| | dilution point, | throughout the suspension. | settled out. |
| Placebo 7 | Fully dispersed, with no | Fully dispersed, with no | Fully dispersed, |
| | particulate matter visible, | particulate matter visible, | with no |
| | | | particulate matter |
| | | | visible. |

### Active and Placebo Formulation Stability Testing in HPMC and Gelatin Capsules

Physical stability of the active and placebo of formulations 1, 6 and 7 in HPMC and gelatin capsules were evaluated under accelerated conditions for 8 weeks. The data suggests that the gelatin shell appears to be more robust than the HPMC shells in all three formulations with no issues associated with the formulations identified in gelatin capsules. Active formulations 6 and 7 perform better than active formulation 1 after 8 weeks' storage under the accelerated conditions.

### Summary of the Results

The evaluation of the initial exemplary formulation has shown that there is potentially an issue with achieving full drug solubilization during manufacture. Several alternative formulations alongside with a formulation that has previously been used for animal toxicity evaluation (which may be unsuitable for human use because of the high labrasol concentration) have been evaluated in this study for drug solubilization during manufacture, the rheological properties, dispersion characteristics and physical stability in gelatin and HPMC capsules. All active formulations 1-10 are believed to provide superior performance by at least some measures as compared to the initial exemplary formulation. Active formulations 1, 6, and 7 showed superior properties as compared to active formulations 2, 3, 4, and 5. Active formulation 6 (comprising 20% API, 69% VitE TPGS and 11% transcutol) appears to be the most promising formulation.

### Example 2A

A formulation containing CORT125281 suitable for oral administration was made which contained 11% CORT125281, 20% Transcutol HP, 30.65% Gelucire, 26.85% Vitamin E, and 11.5% PEG400 (where % indicates % w/w).

### Example 2B

A formulation containing CORT125281 suitable for oral administration was made which contained 11% CORT125281, 10% Transcutol HP, 49% Gelucire 44/14, and 30% Kolliphor (where % indicates % w/w).

### Example 2C

A formulation containing CORT125281 suitable for oral administration was made which contained 11% CORT125281, 29.5% Kolliphor HS15, 39.5% Vitamin E TPGS, and 20% PEG400 (where % indicates % w/w).

### Example 2D

A formulation containing CORT125281 suitable for oral administration was made which contained 11% CORT125281, 35.6% Capryol 90, 26.7% Tween 20, and 26.7% Tween 80 (where % indicates % w/w).

### Example 2E

A formulation containing CORT125281 suitable for oral administration was made which contained 11% CORT125281, 17.8% Medium Chain Triglycerides, 44.5% Tween 20, and 26.7% Kolliphor (where % indicates % w/w).

### Example 2F

A formulation containing CORT125281 suitable for oral administration was made which contained 11% CORT125281, 20% Transcutol HP, and 69% Vitamin E TPGS (where % indicates % w/w). The performance of this formulation was found to be superior in many ways as compared to the performance of some other formulations.

### Example 2G

A formulation containing CORT125281 suitable for oral administration was made which contained 10% CORT125281, 9% Transcutol HP, 27% Kolliphor HS 45, and 54% Labrasol (where % indicates % w/w).

### Example 2H

A formulation containing CORT125281 suitable for oral administration was made which contained 11% CORT125281, 8.9% Transcutol HP, 10% Triacetin, 20% Kolliphor RH40, and 50.1% Gelucire 44/14 (where % indicates % w/w).

### Example 21

A formulation containing CORT125281 suitable for oral administration was made which contained 11% CORT125281, 8.9% Transcutol HP, 21.1% Triacetin, 59% Vitamin E TPGS (where % indicates % w/w).

### Example 2J

A formulation containing CORT125281 suitable for oral administration was made which contained 11% CORT125281, 8.9% Transcutol HP, 11.1% Triacetin, 69% Vitamin E (where % indicates % w/w).

These formulations are believed to be suitable for use in the administration of non-steroidal GRM such as, e.g., CORT125281, to human subjects. For a particular, non-limiting example, the formulation of Example 2F (11% CORT125281, 20% Transcutol HP, and 69% Vitamin E TPGS (where % indicates % w/w) is believed to be suited for use in the administration of CORT125281 to human subjects.

All patents, patent publications, and publications discussed herein are hereby incorporated by reference herein in their entireties.

We claim:

1. A formulation containing CORT125281, the formulation selected from the group of CORT12581 formulations consisting of: a) 11% CORT125281, 69.0% Vitamin E, and 20.0% Transcutol HP; b) 11% CORT125281, 8.9% Transcutol HP, 35.6% Gelucire, 31.2% Vitamin E, and 13.3% PEG400; c) 11% CORT125281, 20% Transcutol HP, 30.65% Gelucire, 26.85% Vitamin E TPGS, and 11.5% PEG400; d) 11% CORT125281, 10% Transcutol HP, 30.0% Kolliphor HS15, and 49.0% Gelucire 44/14; e) 11% CORT125281, 20% PEG400, 39.5% Vitamin E, and 29.5% Kolliphor HS15; f) 11% CORT125281, 35.6% Capryol 90, 26.7% Tween 20, and 26.7% Tween 80; g) 11% CORT125281, 17.8% medium chain triglycerides, 44.5% Tween 20, and 26.7% Kolliphor RH40; h) 10% CORT125281, 9.0% Transcutol, 54.0% Labrasol, and 27.0% Kolliphor HS 15; i) 11% CORT125281, 8.9% Transcutol HP, 20.0% Kolliphor RH40, 50.1% Gelucire 44/14, and 10% Triacetin; j) 11% CORT125281, 59.0% Vitamin E TPGS, 8.9% Transcutol HP, and 21.1% Triacetin; and k) 11% CORT125281, 69.0% Vitamin E TPGS, 8.9% Transcutol HP, and 11.1% Triacetin (wherein “%” is weight % (% w/w)).

2. The formulation of claim 1 which is the formulation a) 11% CORT125281, 69.0% Vitamin E, and 20.0% Transcutol HP (% w/w).

3. The formulation of claim 1 which is the formulation b) 11% CORT125281, 8.9% Transcutol HP, 35.6% Gelucire, 31.2% Vitamin E, and 13.3% PEG400 (wherein “%” is weight % (% w/w)).

4. The formulation of claim 1 which is the formulation c) 11% CORT125281, 20% Transcutol HP, 30.65% Gelucire, 26.85% Vitamin E TPGS, and 11.5% PEG400 (wherein “%” is weight % (% w/w)).

5. The formulation of claim 1 which is the formulation d) 11% CORT125281, 10% Transcutol HP, 30.0% Kolliphor HS15, and 49.0% Gelucire 44/14 (wherein “%” is weight % (% w/w)).

6. The formulation of claim 1 which is the formulation e) 11% CORT125281, 20% PEG400, 39.5% Vitamin E, and 29.5% Kolliphor HS15 (wherein “%” is weight % (% w/w)).

7. The formulation of claim 1 which is the formulation f) 11% CORT125281, 35.6% Capryol 90, 26.7% Tween 20, and 26.7% Tween 80 (wherein “%” is weight % (% w/w)).

8. The formulation of claim 1 which is the formulation g) 11% CORT125281, 17.8% medium chain triglycerides, 44.5% Tween 20, and 26.7% Kolliphor RH40 (wherein “%” is weight % (% w/w)).

9. The formulation of claim 1 which is the formulation h) 10% CORT125281, 9.0% Transcutol, 54.0% Labrasol, and 27.0% Kolliphor HS 15 (wherein “%” is weight % (% w/w)).

10. The formulation of claim 1 which is the formulation i) 11% CORT125281, 8.9% Transcutol HP, 20.0% Kolliphor RH40, 50.1% Gelucire 44/14, and 10% Triacetin (wherein “%” is weight % (% w/w)).

11. The formulation of claim 1 which is the formulation j) 11% CORT125281, 59.0% Vitamin E TPGS, 8.9% Transcutol HP, and 21.1% Triacetin (wherein “%” is weight % (% w/w)).

12. The formulation of claim 1 which is the formulation k) 11% CORT125281, 69.0% Vitamin E TPGS, 8.9% Transcutol HP, and 11.1% Triacetin.

13. A single unit dosage form consisting of a capsule containing between about 10 milligrams (mg) and about 70 mg CORT125281, wherein said capsule has a size selected from size 5, size 4, size 3, size 2, size 1, size 0, size 00, and size 000.

14. A single unit dosage form in the form of a capsule containing a uniform admixture of between about 10 milligrams (mg) and about 70 mg CORT125281 and pharmaceutically acceptable excipients, wherein said uniform admixture is a formulation of claim 1, and wherein said capsule has a size selected from size 5, size 4, size 3, size 2, size 1, size 0, size 00, and size 000.

15. The single unit dosage form of claim 14, wherein the contents of said capsule weigh between about 300 milligrams (mg) and about 600 mg.

16. A method of treating a condition amenable to treatment with a glucocorticoid receptor modulator (GRM) comprising administering the GRM CORT125281 in a single unit dosage form, wherein said single unit dosage form contains a uniform admixture of CORT125281 and pharmaceutical excipients, and wherein said uniform admixture is a formulation of claim 1.

17. The method of claim 16, wherein said single unit dosage form consists of a capsule containing between about 10 milligrams (mg) and about 70 mg of said CORT125281, wherein said capsule has a size selected from size 5, size 4, size 3, size 2, size 1, size 0, size 00, and size 000.

18. The method of claim 16, wherein said condition amenable to treatment with a GRM is selected from Cushing's syndrome, Cushing's Disease, prostate cancer, breast cancer, ovarian cancer, cervical cancer, testicular cancer, endometrial cancer, thyroid cancer, osteosarcoma, pancreatic cancer, fatty liver disease, fibrosis of the liver, cirrhosis of the liver, depression, dementia, a stress disorder, anxiety, and substance abuse disorder.

19. The method of claim 16, wherein said condition amenable to treatment with a GRM is Cushing's Syndrome.

20. A single unit dosage form in the form of a size 0 capsule containing between about 10 milligrams (mg) and about 70 mg CORT125281.
